(19) United States
(12) Patent Application Publication
(10) Pub. No.: US 2023/0123171 A1
(43) Pub. Date: Apr. 20, 2023

(54) DNA RECOMBINASE MEDIATED ASSEMBLY OF DNA LONG ADAPTER SINGLE STRANDED OLIGONUCLEOTIDE (LASSO) PROBES

(71) Applicant: Rutgers, The State University of New Jersey, New Brunswick, NJ (US)

(72) Inventors: Biju Parekkadan, Atlantic Highlands, NJ (US); Lorenzo Tosi, Franklin Park, NJ (US)

(73) Assignee: Rutgers, The State University of New Jersey, New Brunswick, NJ (US)

(21) Appl. No.: 18/046,896

(22) Filed: Oct. 14, 2022

Related U.S. Application Data
(60) Provisional application No. 63/255,509, filed on Oct. 14, 2021.

(51) Int. Cl. C12N 15/10; C12N 15/90

(52) U.S. Cl. CPC C12N 15/102; C12N 15/907; C12N 2800/80

(57) ABSTRACT

Methods of generating mature ssDNA LASSO probes using DNA recombinase mediated assembly are provided. Also provided are mature ssDNA LASSO probes made by the methods, methods of their use, and kits including such.

### CROSS-REFERENCE TO RELATED APPLICATION

This application claims priority to U.S. Provisional Application No. 63/255,509 filed Oct. 14, 2021, herein incorporated by reference in its entirety.

### ACKNOWLEDGMENT OF GOVERNMENT SUPPORT

This invention was made with government support under R01GM127353 awarded by The National Institutes of Health. The government has certain rights in the invention.

### REFERENCE TO AN ELECTRONIC SEQUENCE LISTING

The contents of the electronic sequence listing (sequencelisting.xml; Size: 4,402,907 bytes; and Date of Creation: Oct. 14, 2022) is herein incorporated by reference in its entirety.

### FIELD

This application provides methods of generating mature ssDNA LASSO probes using DNA recombinase mediated assembly. Also provided are mature ssDNA LASSO probes made by the methods, and kits including such.

### BACKGROUND

Long-adapter single-strand oligonucleotide (LASSO) probe libraries enable the massively multiplexed capture of kilobase-sized fragments for downstream sequencing or expression. Mature LASSO probes are single stranded DNA (ssDNA) molecules that become circularized by gap filling and ligation after annealing to target sequences that flank a desired DNA fragment. LASSO probes are a useful tool to capture and clone thousands of kilobase-sized DNA fragments in a single reaction, since they exhibit high specificity and can be massively multiplexed (Tosi et al., Nature BME 2017; 1:0092. doi:10.1038/s41551-017-0092). Because of the large size of the DNA targets (up to 5 KB) that can be captured at single nucleotide resolution, the LASSO probe technology is also a tool for long DNA sequence capture for NGS applications.

Prior LASSO assembly methods (e.g., WO 2016/197065, and FIG. 1B herein) generate DNA side products (such as discordant probes) together with the mature LASSO probes. In addition, LASSO libraries generated from this method contained an unexpectedly large amount of discordant probes resulting from the intermolecular ligation of different LASSO probe precursors during the self-circularization step of the assembly process (Shukor et al., 2019; BMC Biotechnol. 19(1):50), The presence of the discordant probes in the mature LASSO libraries was responsible for a significant reduction of the capture efficiency and the production of undesirable low molecular weight unspecific DNA amplicons in post capture PCR. Moreover, the previous LASSO assembly method used two consecutive PCR steps that introduce several different DNA artifacts in the final LASSO libraries, such as DNA polymerase errors, skewing the distribution of PCR products due to unequal amplification of different probes, probe-probe fusion products, accumulation of primers dimers.

These drawbacks have limited the use of mature LASSO probe to simple genomes, like bacteria. For highly complex eukaryotic genomes, such as a human genome, a higher capture efficiency and higher purity of the mature LASSO probe library is needed.

The new methods provided herein address these issues, as the methods avoid the self-circularization step of the previous LASSO assembly process and the initial fusion PCR steps. This results in a pure population of mature LASSO probes with a significant improvement in the capture efficiency.

### SUMMARY

Provided herein are single stranded (ss) DNA Long Adapter Single Stranded Oligonucleotide (LASSO) probes, methods of making such, and methods of their use. In one example, the DNA LASSO probes include, from 5′ to 3′, (1) a ligation arm sequence complementary to a 5′ region of a target sequence, (2) a backbone sequence that is not complementary to the target sequence, and comprises a recombination site, and (3) an extension arm sequence complementary to a 3′ region of the target sequence, wherein the ligation arm sequence and extension arm sequence are complementary to 5′ and 3′ regions of a single target sequence, respectively. In some examples the ligation arm sequence is at least 20 nucleotides (nt), such as 20-40 nt, 20-50 nt, or 20-80 nt, the backbone sequence is at least 100 nt, such as at least 200, at least 300, at least 350 nt, or at least 400 nt, such as 200 to 2500, 200-500, 200-2000, 200-2500, 200-1500, 200-1000, 200-800 nt, 200-400 nt, 300 to 400 nt, 350 to 450 nt, or 250-300 nt, the extension arm sequence at least 20 nt, such as 20-80 nt or 20-40 nt, or combinations thereof. In some examples, the 5′ and 3′ regions of the target sequence to which the ligation and extension arms hybridize are at least 200 nt apart, such as at least 500, at least 1000, at least 5,000, at least 10,000, at least 20,000, or at least 30,000 nt apart, such as 200-30,000 nt apart on the target sequence. In some examples, the melting temperature (Tm) of the extension arm is 65-70° C. and ligation arm is 70-75° C. In some examples, the Tm of the ligation arm is about 5° C. higher than the extension arm. In some examples, the Tm of the extension arm and the ligation arm are in the same range, such as 65-70° C. for both, or have the same Tm, such as 65° C.

Compositions that include one or more of the disclosed ssDNA LASSOs probes are also provided, and can include other materials, such as a pharmaceutically acceptable carrier (e.g., water or saline). Kits that include one or more of the disclosed ssDNA LASSOs probes (such as a library of mature ss DNA LASSO probes, such as a custom library or a general purpose library e.g. human oncogene panel) are also provided, and can include other materials, such as and one or more endonucleases, one or more exonucleases, one or more polymerases (such as a DNA polymerase, such as one having low strand displacement, such as Kapa HiFi), one or more ligases, one or more recombinases, one or more reagents for PCR, or combinations thereof. In specific examples, the kit includes one or more of the disclosed ssDNA LASSOs probes (such as a probe library), and one or more of a gap filling mix (e.g., a thermostable DNA ligase, a DNA polymerase [such as one having low strand displacement, such as Kapa HiFi], dNTPs, glycerol, buffer), linear DNA digestion solution (e.g., Exonucleases I, III and Lambda, buffer and glycerol), oligonucleotide primers for post capture PCR reaction, post capture PCR master mix (e.g., DNA polymerase, dNTPs and buffer), and a positive control for the capture reaction (e.g., a LASSO probe that captures 1 kb target sequence within the genome of the phage M13mp18 single stranded DNA, or the LASSO probe and an aliquot of M13mp18 single stranded DNA (New England Biolab N4040S)).

Also provided are methods of generating the disclosed ssDNA LASSO probes. In some examples, such a method includes providing a double stranded pre-LASSO probe (which can be generated from a ssDNA pre-LASSO probe, such as any of SEQ ID NOS 1-3088, 3090-3093, 3117-3121, and 3126). In some examples, the ssDNA pre-LASSO probe used to generate the double stranded pre-LASSO probe comprises at least 80%, at least 85%, at least 90%, at least 95%, at least 98%, at least 99%, or 100% sequence identity to any of SEQ ID NOS: 1-3088, 3090-3093, 3117-3121 and 3126. The dsDNA pre-LASSO probes include from 5′ to 3′ (i) a first primer annealing site sequence, (ii) an extension arm sequence, (iii) an inverted PCR primer annealing site comprising a restriction site that allows for asymmetric cutting, (iv) a ligation arm sequence, and (v) a second primer annealing site sequence. The ds pre-LASSO probe is contacted with a double stranded linear pLASSO vector comprising from 5′ to 3′ (e.g., “a” in pLASSO 14 FIG. 2A, 2B is the 5′ end) (i) the second primer annealing site sequence (i.e., the second primer annealing site sequence of the pre-LASSO probe), (ii) a first backbone region that does not substantially hybridize to the target sequence, (iii) a first recombination site, (iv) a selectable marker, (v) an origin of replication, (vi) a second recombination site, (vii) a second backbone region that does not substantially hybridize to the target sequence and (viii) the first primer annealing site sequence (i.e., the first primer annealing site sequence of the pre-LASSO probe), wherein the double stranded linear pLASSO vector further includes a nicking endonuclease recognition site (for example in the backbone), a restriction site not in the backbone (for example between the first recombination site and the selectable marker) used to digest a plasmid (e.g., SwaI), and optionally a first restriction endonuclease site (such as SalI) and an optional second restriction endonuclease site (such as BamHI) (wherein the optional first and second restriction endonuclease sites can be used to ensure cloning of a pre-LASSO probe into a linear pLASSO vector was successful; in some examples these are in the backbone region), under conditions to allow annealing, gap filling and ligation of the first and second primer annealing sites of the pre-LASSO probe to the first and second primer annealing sites of the linear pLASSO vector, thereby generating a circular pLASSO vector containing the pre-LASSO probe; introducing the circular pLASSO vector into host cells, thereby generating transformed host cells comprising the circular pLASSO vector; growing the transformed host cells in the presence of a growth media comprising reagents that do not permit growth of the host cells in the absence of the selectable marker; extracting the circular pLASSO vector from the transformed host cells; contacting the extracted circular pLASSO vector with a nicking endonuclease specific for the nicking endonuclease recognition site, under conditions that cleave one nucleic acid strand of the extracted circular pLASSO vector, thereby producing a relaxed circular pLASSO vector; contacting the relaxed circular pLASSO vector with a recombinase specific for the first and second recombination site, under conditions that recombination of the relaxed circular pLASSO vector occurs, thereby generating (i) a plasmid comprising the restriction site (e.g., SwaI in FIG. 1A), a recombination site, the selection marker, and the origin of replication and (ii) a minicircle comprising the double stranded pre-LASSO probe, the first and second backbones, and 50% of each recombination site (e.g., if the recombination sites in pLASSO are AA and BB, after the recombination they become AB and AB); digesting the plasmid with a restriction enzyme (such as SwaI or other restriction enzyme) and exonuclease V; using inverted PCR to linearize the minicircle, using a first primer and a second primer that hybridize to the inverted PCR primer annealing site, wherein the first primer includes a restriction enzyme site (e.g., a Type IIS (shifted cleavage) restriction enzyme, such as BspQI, BsaI, BsmBI, BbsI, Esp3I, BtgZI, BspMI, BsmFI, SapI restriction enzyme site, which recognize an asymmetric DNA sequence and cleaves outside its recognition site located in the inverted PCR primer annealing site 54 and cleaves the 3′-5′(bottom strand) a DNA strand exactly at the 5′ end of the extension arm. while the 5′-3′ DNA strand (top strand) is cut inside the “inverted PCR primer annealing site) and wherein the second primer comprises a 3′-uracil and the three 5′-end nt are modified nucleotides resistant to exonuclease treatment (e.g., connected by phosphorothioate bonds that are resistant to lambda exonuclease treatment, such as 5′ A*T*C*GCCGCAAGAAGTGTU 3′; SEQ ID NO: 3105 thereby generating a linear double stranded minicircle with a 5′ end and 3′ end, wherein the 5′ end of the linear double stranded minicircle is the first primer annealing site at the 3′ end of the linear double stranded minicircle is the second primer annealing site; removing all or part of the first and second primer annealing sites from the 5′ and 3′ end of the linear double stranded minicircle by restriction digestion and/or glycosylase digestion to produce a digested linear double stranded minicircle, and removing one of the two strands of the digested linear double stranded minicircle, thereby producing the ssDNA LASSO probe.

Also provided are methods of using in the disclosed ssDNA LASSO probes. In some examples, the methods include detecting a target nucleic acid sequence. Such methods can include contacting a sample comprising the target sequence with one or more ssDNA LASSO probes provided herein, wherein the ligation arm sequence and the extension arm sequence are complimentary to a 5′ region of the target sequence and to a 3′ region of the target sequence, respectively; hybridizing the ligation arm sequence and extension arm sequence to the target sequence; gap filling to copy the target sequence between the ligation arm sequence and extension arm sequence using a polymerase (such as a DNA polymerase, such as one having low strand displacement, such as Kapa HiFi), thereby generating a ssDNA circle containing a copy the targeted DNA sequence; ligating the resulting molecule, thereby generating a circular single stranded DNA fragment comprising the target sequence; isolating the circular single-stranded DNA fragment comprising the target sequence (e.g., optionally by digesting linear DNA in the sample, for example by adding directly to the capture reaction an aliquot of “linear DNA digestion solution” containing Exonuclease I, Exonuclease III and Lambda Exonuclease); and amplifying the circular single stranded DNA fragment comprising the target sequences, thereby detecting the target sequences (for example by detecting expected size DNA target sequence amplicons, e.g., using gel electrophoresis or the Bioanalizer). Also provided are libraries of target sequences generated by such a method.

Also provided are kits that include (a) a double stranded pre-LASSO probe comprising from 5′ to 3′(i) a first primer annealing site sequence, (ii) the extension arm sequence, (iii) an inverted PCR primer annealing site comprising a restriction site that allows for asymmetric cutting, (iv) the ligation arm sequence, and (v) a second primer annealing site sequence, (b) a double stranded linear pLASSO vector comprising from 5′ to 3′ (i) the second primer annealing site sequence (ii) a first backbone region that does not substantially hybridize to the target sequence, (iii) a first recombination site, (iv) a selectable marker, (v) an origin of replication, (vi) a second recombination site, (vii) a second backbone region that does not substantially hybridize to the target sequence, and (viii) the first primer annealing site sequence, wherein the double stranded linear pLASSO vector further includes a nicking endonuclease recognition site (for example in the backbone), a restriction site not in the backbone (for example between the first recombination site and the selectable marker) used to digest a plasmid (e.g., SwaI or other restriction enzyme), and optionally a first restriction endonuclease site (such as SalI) and an optional second restriction endonuclease site (such as BamHI); and (c) optionally one or more endonucleases, one or more exonucleases, one or more recombinases; one or more growth media; one or more reagents for inverted PCR, or combinations thereof.

Also provided are isolated nucleic acid molecules, such as a pre-LASSO probe, such as one including at least 80%, at least 85%, at least 90%, at least 95%, at least 98%, at least 99%, or 100% sequence identity to any of SEQ ID NOS: 1-3088, 3090-3093, 3117-3121, and 3126. Also provided are vectors which include such probes. Also provided are isolated cells that include such isolated nucleic acid molecules or vectors, including prokaryotic or eukaryotic cells, such as bacterial, yeast, or mammalian cells.

The foregoing and other objects and features of the disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic drawing providing an overview of one embodiment of the disclosed DNA recombinase mediated assembly method for making mature ssDNA LASSO probes 30.

FIG. 1B is a schematic drawing providing an overview of the previous long adapter based assembly method for making mature ssDNA LASSO probes.

FIG. 2A is a schematic drawing providing details of an exemplary pre-lasso probe 12, pLASSO vector 14 (5′-end is “a”), and ssDNA mature LASSO probe 30.

FIG. 2B is a schematic drawing providing details on the linearization of the pLASSO vector 14 using tailed linearization primers 15 containing a and b selector sequences.

FIG. 2C is a schematic drawing providing details of an exemplary pLASSO vector 14 with nucleotide lengths provided.

FIG. 2D is a schematic drawing providing details of an exemplary pre-LASSO probe 12 sequence (SEQ ID NO: 3126). The ligation arms and extension arm are variable. Nucleotides (nt) 1-21 primer selector F annealing site; nt 1-18 primer selector a; nt 25-58 ligation arm; nt 59-121 inverted PCR primer annealing sites, nt 59-76 primer Thiol R (contains 3 phosphorothioated bonds at the 3′end); nt 77-95 Primer Sap1F (contains the restriction endonuclease site SapI); nt 96-121 Extension arm (variable), nt 122-141 Primer Selector R annealing site.

FIG. 2E is a schematic drawing providing details of an exemplary mature LASSO probe 30 sequence (GAGGGATTGGGCGTCAACGGGCAGTAGGATCCTACGGTCATTCAGCCTCCC CTTCTCCTGGTACGGAAGCAAAGCCTATGTTAAACACTGACTATCTGAAGCT CTCCTTCCCTGAAGGCTTGAGAGATTCATGAACTTCGAGGAAGGACGGAGA GTTTATTTATAAGGAACCAACTTCCCCTCCGATGGCCCTGTCATGAATTCTC ATGTTTGACAGCTTATCATCGATAAGCTTCCCATGGATAACTTCGTATAATG TATGCTATACGAAGTTATGGCTCGAGGAATTCAGAGAAGTCATCAAAGAGT TTAAAGAGTTTATGAGATTTAAGGTCAAGACAACGAGACACGAGTTCGAGA TTGAGGGAGAGAAGGCCCCTCAGCGGCCTTATAACTATAACGGTCCTAAGG TAGCGAACGAACAAACCGCTAAGCTCAAGGTCACAAAAGCAGACGACGGC CAGTGTCGACATGTCACTGTATCGCCGTCTAGTTCTGCTGTCTTGTC (SEQ ID NO: 3112). Nucleotides (nt) 1-26 extension arm (variable), nt 27-46 primer selector R annealing site; nt 47-207 backbone; 47-75 pLASSO linearization primer F annealing site; nt 52-72 postCaptR primer; nt 245-278 Loxp; nt 282-452 backbone; nt 380-386 Nt.BvCl site; nt 428-452 pLASSO linearization primer F annealing site; nt 428-448 postCaptF primer; nt 453-473 Primer selector F annealing site, nt 474-508 ligation arm (variable).

FIGS. 3A-3B is a schematic drawing providing details of the previous long adapter based assembly method, (A) resulting mature LASSO probe, and (B) LASSO probe precursors pre-LASSO and long adapter.

FIG. 4 is a digital image showing amplification of a pre-LASSO probe in lane 2 (lane 1 is a ladder).

FIG. 5 is a digital image showing amplification of pLox2+ (1); L=ladder.

FIG. 6 is a digital image showing digestion of a correctly assembled pLASSO, as indicated by digestion with (1) SwaI, (2) EcoRI, (3) EcoR1 plus Swa1, or (4) undigested. L=ladder.

FIG. 7 is a digital image showing amplification of a linearized pLASSO with the correct size (3.3 kb) in lane 2 (lane 1 is a ladder).

FIG. 8 is a digital image showing successful cloning of a pre-LASSO pool into pLASSO. A ˜160 bp band is present (1). L1 and L2 are ladders.

FIG. 9 is a digital image showing amplification of a mature LASSO probe (˜550 bp). L=ladder.

FIGS. 10A-10C is a schematic drawing of an exemplary embodiment of the disclosed DNA recombinase mediated LASSO assembly methods (A) A single pre-LASSO probe or a pre-LASSO library in shuttled in the linearized pLASSO vector via a Gibson Assembly reaction and used for transformation in E. coli. The coned library is harvested by scraping a sufficient number of colonies from plates. Plasmids are purified by using a plasmid miniprep. The presence of the pre-LASSO probes in the plasmids was verified by digesting with restriction enzymes that cut adjacently to the Gibson assembly insertion sites (Sal1, BamH1 sites). Gel electrophoresis results illustrate successful cloning of the pre-LASSO library in pLASSO. (B) The native supercoiled plasmids obtained by colony miniprep, are converted in the relaxed form by nicking with endonuclease Nt.BspQ1 that uses a recognition site located in the primer annealing site of the inserted pre-LASSO probe. Cre recombination of the LoxP sites produces a DNA minicircle containing the pre-LASSO and a circular 2.7 kb DNA circle, the remaining part of pLASSO. After recombination, the 2.7 kb DNA circle, together with the unreacted plasmids and bigger DNA circles generated by inter-plasmid recombination are eliminated by restriction followed by exonuclease digestion. Gel electrophoresis results illustrate successful formation of the expected nicked DNA minicircles (orange arrow) together with the 2.7 kb circular DNA remaining parts of pLASSO (green arrow), the unreacted plasmid (blue arrow). The approximately 6 kb band (yellow arrow) correspond to the recombination of two different plasmids (inter-plasmid recombination). Legend. Sal1, BamH1 and BspQ1 indicate restriction enzyme sites; Nick indicates nicking endonuclease site NtBspQ1; * indicates phosphorothioate bonds, U indicate a deoxyuracil moiety. Gel 1: L1. 1 kb DNA Ladder (NEB), L2. Low MW DNA Ladder (NEB), Lane 1. pLASSO library digested with Sal1 and BamH1, Lane 2. Negative control: pLASSO alone. Orange arrow. excited preLASSO probes inserts. Gel 2: L1. 1 kb DNA Ladder (NEB), L2. Low MW DNA Ladder (NEB), Lane 1. Cre recombination of nicked pLASSO library, Lane 2. Cre recombination of unnicked pLASSO library Orange arrow. DNA Minicircle containing preLASSO probes. Green arrow. Circular remaining part of pLASSO. Blue arrow. Unreacted pLASSO library Yellow arrow. Circular fusion products generated by intermolecular recombination events of two pLASSO plasmids. Gel 3: L2. Low MW DNA Ladder (NEB) Lane 1. Inverted PCR product derived from the linearization of the DNA minicircle Lane 2. Negative control: Cre-recombinase was not added in (b) consequently the DNA minicircle was not formed and the pLASSO library was completely destroyed during restriction/digestion step.

FIG. 11: Gel electrophoresis of post capture PCR amplicons obtained by capturing a single 1 kb target sequence in a constant Human total genomic DNA background (800 human genomes/μl). Captures displayed the 16 lanes were performed by testing tenfold dilutions of the LASSO probe against tenfold dilutions of the target sequence according with the concentrations shown in the table. In lane 12 no signal because pipetting error. Lane 16 negative control of the capture.

FIGS. 12A-12D. Workflow of LASSO probe library assembly and capture using (A) the novel DNA recombinase mediated methodology or (B) the previous intramolecular ligation assembly methodology in capturing a library of kilobase-sized ORFs from E. coli genomic DNA. The pre-LASSO probe pools are converted in a mature LASSO probe pool stepwise in a pooled format. Thousands of LASSO probes are hybridized on target DNA. Closed DNA circles containing captured ORFs are selected by exonuclease digestion, and then PCR amplified using universal primers. (C) Probe assembly NGS data analysis. (D) Mean read depth of all sequencing reads mapped to the LASSO probe libraries. The reference probe library sequences (N=3164) were grouped according to ranges of expected capture size in increasing order to highlight biases in probe formation and predict downstream capture performance. Read depth is defined as the number of reads that map to a specific reference sequence. On the horizontal axis, probe library sequences were grouped according to expected probe capture size ranges. The percentages of ORFs represented by concordant probes within these expected capture size ranges were plotted for both LASSO assembly methods. Concordant probes are properly formed probes with paired-end reads that map to a unique probe reference sequence.

FIGS. 13A-13F: (A) Average “arm concordancy” indicates the average of correctly paired probe arms versus total read sequences per probe type in the LASSO probe library obtained by using the DNA Recombinase Mediated Assembly (blue) or the previous methodology developed by Tosi et al. (2017) by using 2 ml ligation volume (red) and 50 μl volume (gray) for LASSO probe assembly. (B) Plot of absolute count of concordant LASSO probe types. (C) Median RPKM enrichment ratios of targeted ORFs versus non-targeted genetic elements ratios of a LASSO probe library obtained by using the DNA Recombinase Mediated Assembly (black) and the assembly method developed by Tosi et al. (2017) (gray). (D) Post-capture PCR of circles obtained from the capture of 3,078 ORFs of E. coli K12 performed using the LASSO probe library obtained with the DNA recombinase mediated assembly. The inset is a histogram denoting the size distribution of the targeted ORFs split into bin sizes of 40 bp. Targeted ORFs have an increase in 140 bp of residual LASSO sequences once captured and run on a gel. (E) Bee swarm plot combined with boxplot Average depth of sequencing per kilobase for each targeted ORF (n=3095) and non targeted ORF (n=905). Center lines show the medians; box limits indicate the 25th and 75th percentiles as determined by R software; whiskers extend 1.5 times the interquartile range from the 25th and 75th percentiles, outliers are represented by dots. n=3057, 1004 sample points. F. Normalized read depth of targeted ORFs as a function of the length of the ORFs.

FIGS. 14A-14B. Reagent optimization (A) Effect of type of DNA polymerase on capture efficiency KAPA Hi Fi v/s Omni Klentaq LA; (B) Effect of ligase concentration on capture efficiency.

FIGS. 15A-15D. (A) Effect of different melting temperature (Tm) ligation arms (65° C., 70° C. and 75° C.) and DNA backbone lengths (350 and 701 bp) in capturing a 3 kb target sequence within the M13 bacteriophage genomic DNA. Capture efficiency is expressed as total nanograms of post capture PCR product obtained by Gel Analyzer quantification. (B) Gel electrophoresis showing DNA target band intensity following PCR post-capture with LASSOs with different linker length: lane 1 to 5 show targets captured with a shorter 350 bp backbone linker, lane 6 to 10 show targets captured with a longer 701 bp backbone linker and with different melting arm temperature: lane 2, 3 and 4 capture of a 3K DNA target with LASSOs having 65° C., 70° C. and 75° C. ligation arms melting temperature respectively, lane 7, 8 and 9 capture of a 3K DNA target with LASSOs having 65° C., 70° C. and 75° C. ligation arms melting temperature respectively. Lane 1 and 6 capture of 1 kb DNA target (positive control). Lane 5 to 10 are negative controls identical to 1 and 6 but without template DNA. (C) Gel electrophoresis of post capture PCR amplicons of DNA target sequences within the M13 bacteriophage genomic DNA. Lane 1 and lane 4, capture of 1 kb DNA target (positive control). Lane 2 capture of a 4 kb DNA target, lane 3 and lane 6 negative control (identical to lane 1 but no DNA ligase in the gap filling mix), lane 5 capture of a 5 kb DNA target. (D) Sanger sequencing analysis of the 5 kb amplicon. The top inset shows the backbone sequence, the ligation arm of the LASSO probe and the initial part of the target sequence. The bottom insert shows the end of the backbone sequence, the extension arm of the LASSO probe and the end of the 5 kb target sequence. SEQ ID NOS: 3124 and 3125.

FIGS. 16A-16C. (A) Distribution of target lengths of sub pools. ligation arm Tm 65-70° C. extension arm Tm 70-75° C. (L65E70), ligation arm Tm 60-65° C. extension arm Tm 70-75° C. (L60E70), ligation arm Tm 70-75° C. extension arm Tm 65-70° C. (L70E65), ligation arm Tm 70-75° C. extension arm Tm 60-65° C. (L70E60), extension and ligation arm in the same range 65-70° C. (L65E65) respectively. (B) Table showing melting temperature intervals of probe arms for the lasso probe sub pools and number of LASSO probes in the sub pools. (C) Gel electrophoresis of post capture amplicons obtained by capturing ORFs from E. coli k12 genome using the LASSO subpools. Lane 1 capture with LASSO probes having low ligation arm melting temperature (65-70), lane 2 capture with LASSO probes having very low ligation arm melting temperature (60-65), lane 3 capture with LASSO probes having low extension arm melting temperature (65-70), lane 4 capture with LASSO probes having very low extension arm melting temperature (60-65), lane 5 capture with LASSO probes having extension and ligation arm in the same range (65-70).

FIGS. 17A-17D. (A) Bean plot representing the coverage for each targeted sequence in the different LASSO probe pools. Pools have different melting temperature of the capture arms as follow: ligation arm Tm 65-70° C. extension arm Tm 70-75° C. (L65E70), ligation arm Tm 60-65° C. extension arm Tm 70-75° C. (L60E70), ligation arm Tm 70-75° C. extension arm Tm 65-70° C. (L70E65), ligation arm Tm 70-75° C. extension arm Tm 60-65° C. (L70E60), extension and ligation arm in the same range 65-70° C. (L65E65) (B) Bean plot representing the coverage for targeted sequences of the pools listed in (A) cloned into pDONR, (C) Coverage distribution of non targeted ORFs in each of the same pools listed in (A). Black lines show the medians for each pools; white lines represent individual data points; polygons represent the estimated density of the data. (D) Density plot showing the distribution of sequences of the extension and ligation arm in the same range (L65E65) pool according the difference in the arm melting temperature as represented on the x-axis (ΔTm=Tm extension arm−Tm ligation arm).

### SEQUENCE LISTING

The nucleic acid sequences listed in the accompanying sequence listing are shown using standard letter abbreviations for nucleotide bases as defined in 37 C.F.R. 1.822. Only one strand of each nucleic acid sequence is shown, but the complementary strand is understood as included by any reference to the displayed strand. All strands are shown 5′ to 3′ unless otherwise indicated. The Sequence Listing is submitted as an XML file, “Sequence Listing.xml,” created on Oct. 14, 2022, 4,402,907 bytes, which is incorporated by reference herein.

SEQ ID NOS: 1 to 3088 provide exemplary pre-Lasso nucleic acid sequences.

SEQ ID NO: 3089 is an exemplary EcoRI backbone sequence.

SEQ ID NO: 3090 is an exemplary pre-LASSO probe sequence, wherein the N at nt 22 is a ligation arm, and nt 60 is an extension arm, wherein the sequence of the ligation arm and extension arm depend on the target sequence. Nt 1-21 is the primer selector F annealing site, nt 23-59 is the inverted PCT primer annealing site, and nt 61-80 the primer selector F annealing site.

SEQ ID NO: 3091 is an exemplary pre-LASSO M13 probe sequence. Nt 1-21 is the primer selector F annealing site, nt 22-47 is the ligation arm, nt 48-84 is the inverted PCT primer annealing site, mt 85-109 the extension arm, and nt 110-129 the primer selector F annealing site.

SEQ ID NO: 3092 is an exemplary pre-LASSO GAPDH probe sequence. Nt 1-21 is the primer selector F annealing site, nt 22-51 is the ligation arm, nt 52-88 is the inverted PCT primer annealing site, nt 89-115 the extension arm, and nt 116-135 the primer selector F annealing site.

SEQ ID NO: 3093 is an exemplary pre-LASSO F-actin probe sequence. Nt 1-21 is the primer selector F annealing site, nt 22-46 is the ligation arm, nt 47-83 is the inverted PCT primer annealing site, nt 84-108 the extension arm, and nt 109-128 the primer selector F annealing site.

SEQ ID NO: 3094-3101 are exemplary selector sequences.

SEQ ID NO: 3102 is a pLASSO linearization a sequence.

SEQ ID NO: 3103 is a pLASSO linearization b sequence.

SEQ ID NO: 3104 is a Sap1F primer sequence.

SEQ ID NO: 3105 is the sequence for the ThiolR primer.

SEQ ID NO: 3106 is an exemplary sequence for reserve primer PostCaptR.

SEQ ID NO: 3107 is an exemplary sequence for forward primer PostCaptF.

SEQ ID NO: 3108 is an exemplary sequence for forward primer Neb1F.

SEQ ID NO: 3109 is an exemplary sequence for reverse primer Neb1R SEQ ID NO: 3110 is an exemplary sequence for forward primer AttB1 CapF.

SEQ ID NO: 3111 is an exemplary sequence for reverse primer AttB2 CapR.

SEQ ID NO: 3112 is the sequence for an exemplary mature LASSO probe 30.

SEQ ID NO: 3113 is the sequence for primer selector F annealing site 1.

SEQ ID NO: 3114 is the sequence for primer selector R annealing site 2.

SEQ ID NO: 3115 is the inverted PCR primer annealing site.

SEQ ID NO: 3116 is an exemplary target sequence.

SEQ ID NO: 3117 is the pre-LASSO 3 kb M13 for 65° C. sequence.

SEQ ID NO: 3118 is the pre-LASSO 3 kb M13 for 70° C. sequence.

SEQ ID NO: 3119 is the pre-LASSO 3 kb M13 for 75° C. sequence.

SEQ ID NO: 3120 is the pre-LASSO 4 kb M13 sequence.

SEQ ID NO: 3121 is the pre-LASSO 5 kb M13 sequence.

SEQ ID NO: 3122 is the 350 bp EcoR1 Backbone sequence.

SEQ ID NO: 3123 is the 700 bp EcoR1 Backbone sequence.

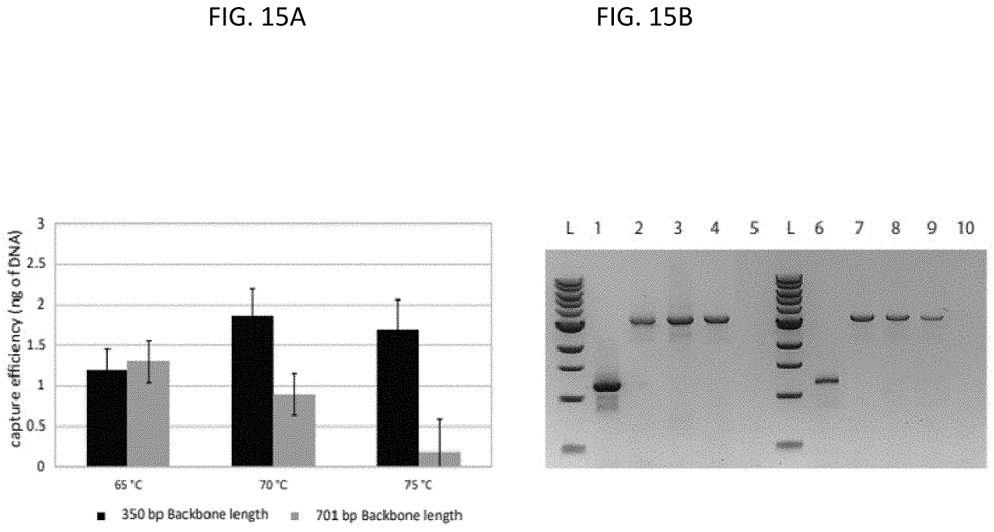

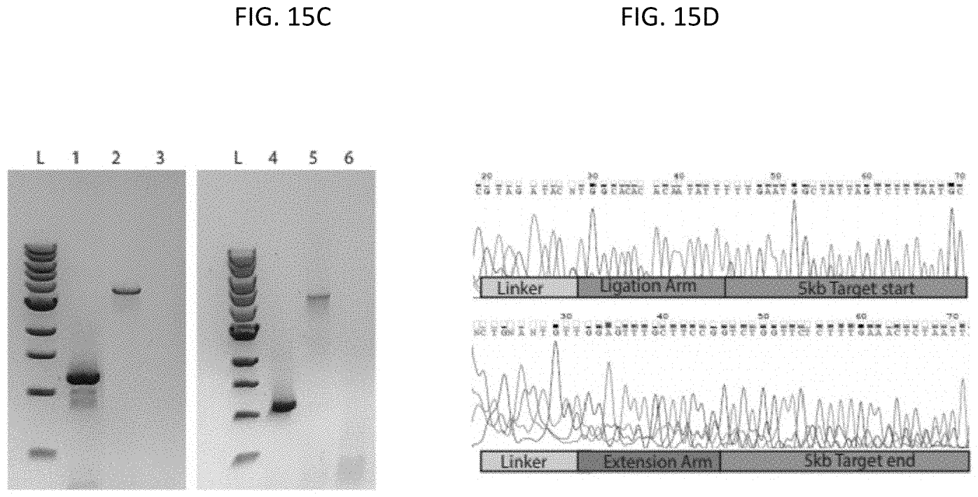

SEQ ID NOS: 3124 and 3125 are the Sanger sequenced for the 5 kB target shown in FIG. 15D.

SEQ ID NO: 3126 is an exemplary pre-LASSO sequence.

cAgACGACGGCCAGTgtcgacATGTCACTGTATCGCCGTCTAGTTCTGC
TGTCTTGTCAACACTTCTTGCGGCGATGGTTCCTGGCTCTTCGATCGAG
GGATTGGGCGTCAACGGGCAGTAGGATCCTACggtcATtCAGC

### DETAILED DESCRIPTION

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All Genbank® Accession numbers (the sequence available on Oct. 14, 2020) mentioned herein are incorporated by reference in their entireties. The materials, methods, and examples are illustrative only and not intended to be limiting.

In order to facilitate review of the various embodiments of the disclosure, the following explanations of specific terms are provided:

cDNA (complementary DNA): A piece of DNA lacking internal, non-coding segments (introns) and regulatory sequences which determine transcription. cDNA can be synthesized in the laboratory by reverse transcription from messenger RNA extracted from cells.

Culture or growth media: Any substance used to culture cells, such as mammalian cells and microorganisms, for example bacteria. Such media includes any growth medium (e.g., broth or gel) which supports life (e.g., a microorganism that is actively metabolizing carbon). Culture medium usually contains a carbon source, such as glucose, xylose, cellulosic material and the like. The carbon source can be anything that can be utilized, with or without additional enzymes, by the cell or microorganism for energy.

Gene: A part of a genome, or a nucleic acid molecule, comprising transcriptional and/or translational regulatory sequences and/or a coding region and/or non-translated sequences (e.g., introns, 5′- and 3′-untranslated sequences). The coding region of a gene (such as a target gene) may be a nucleotide sequence coding for an amino acid sequence or a functional RNA. Genes include regulatory sequences (e.g. promoters, enhancers, etc.) and/or intron sequences, and a sequence, termed an “open reading frame” that encodes a protein.

Hybridization: To form base pairs between complementary regions of two strands of DNA, RNA, or between DNA and RNA, thereby forming a duplex molecule.

Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (Allows Sequences that Share at Least 90% Sequence Identity to Hybridize to One Another)
Hybridization: 5×SSC at 65° C. for 16 hours
Wash twice: 2×SSC at room temperature (RT) for 15 minutes each
Wash twice: 0.5×SSC at 65° C. for 20 minutes each

High Stringency (Allows Sequences that Share at Least 80% Sequence Identity to Hybridize to One Another)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each

Low Stringency (Allows Sequences that Share at Least 60% Sequence Identity to Hybridize to One Another)
Hybridization: 6×SSC at RT to 55° C. for 16-20 hours
Wash at least twice: 2×-3×SSC at RT to 55° C. for 20-30 minutes each.

Isolated: An “isolated” biological component (such as a nucleic acid molecule, protein, or cell) has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins.

Mammal: This term includes both human and non-human mammals. Examples of mammals include, but are not limited to: humans, non-human primates, pigs, cows, goats, cats, dogs, rabbits, rats, and mice. In one example, a target sequence is a mammalian nucleic acid molecule, such as a mammalian gene or cDNA.

Nucleic Acid Molecule: Refers to DNA and RNA molecules, such as cDNA and mRNA. Can include naturally occurring and/or non-naturally occurring nucleotides.

Nucleotides: The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Includes nucleotides containing modified bases, modified sugar moieties and modified phosphate backbones, for example as described in U.S. Pat. No. 5,866,336 to Nazarenko et al. (herein incorporated by reference). Examples of modified sugar moieties which may be used to modify nucleotides at any position on its structure include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

ORF (open reading frame): A series of nucleotide triplets (codons) coding for amino acids without any termination codons. These sequences are usually translatable into a peptide.

Pharmaceutically Acceptable Carrier: The pharmaceutically acceptable carriers useful in this disclosure are conventional. Remington's Pharmaceutical Sciences, by E. W. Martin, Mack Publishing Co., Easton, Pa., 19th Edition (1995), describes examples of such that can be used with one or more nucleic acid molecules provided herein. Examples include pharmaceutically and physiologically acceptable fluids such as water, physiological saline, balanced salt solutions, aqueous dextrose, glycerol or the like.

Polymerase Chain Reaction (PCR): An in vitro amplification technique that increases the number of copies of a nucleic acid molecule (for example, a nucleic acid minicircle). The product of a PCR can be characterized by techniques such as electrophoresis, restriction endonuclease cleavage patterns, oligonucleotide hybridization or ligation, and/or nucleic acid sequencing. A specific type of PCR is inverse PCR, which is used to amply DNA with only one known sequence.

In some examples, PCR utilizes primers, for example, DNA oligonucleotides 10-100 nucleotides in length, such as about 15, 20, 25, 30 or 50 nucleotides or more in length (such as primers that can be annealed to a complementary target DNA strand by nucleic acid hybridization to form a hybrid between the primer and the target DNA strand). Primers can be at least 15, at least 20, at least 25, at least 30, at least 35, at least 40, at least 45, at least 50 or more consecutive nucleotides of a nucleotide sequence of interest. Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990).

Primer: Short nucleic acids, for example DNA or RNA oligonucleotides 10 nucleotides or more in length, which are annealed to a complementary target nucleic acid strand (e.g., a minicircle nucleic acid molecule) by nucleic acid hybridization to form a hybrid between the primer and the target nucleic acid strand, then extended along the target nucleic acid strand by a polymerase enzyme. Individual primers can be used for nucleic acid sequencing. In addition, primer pairs can be used for amplification of a nucleic acid sequence, e.g., by PCR (such as inverse PCR) or other nucleic-acid amplification methods.

Primers can have at least 10 nucleotides complementary to the nucleic acid molecule to be sequenced. To enhance specificity, longer primers can be employed, such as primers having at least 15, at least 20, at least 30, at least 40, at least 50, at least 60, at least 70, at least 80, at least 90 or at least 100 consecutive nucleotides of the complementary nucleic acid molecule to be sequenced. Methods for preparing and using primers are described in, for example, Sambrook et al. (1989) Molecular Cloning: A Laboratory Manual, Cold Spring Harbor, N.Y.; Ausubel et al. (1987) Current Protocols in Molecular Biology, Greene Publ. Assoc. & Wiley-Intersciences.

In one example, a primer is a DNA, RNA, or a mixture of both.

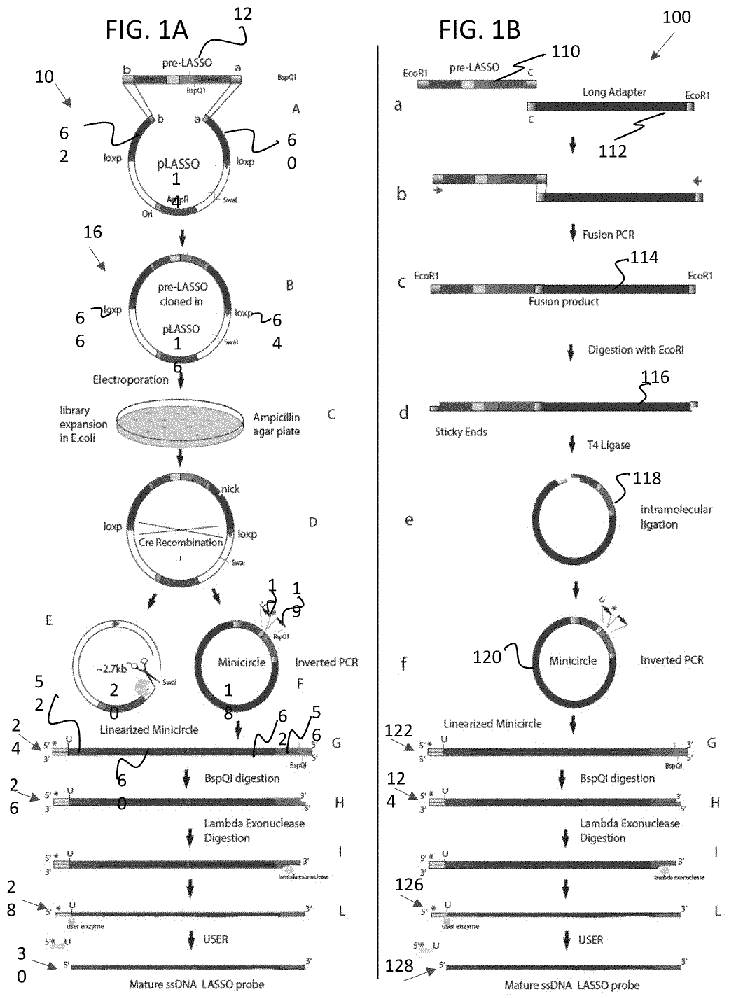

Recombinant: A recombinant nucleic acid is one that has a sequence that is not naturally occurring or has a sequence that is made by an artificial combination of two otherwise separated segments of sequence. In some examples artificial combination is accomplished by chemical synthesis or by the artificial manipulation of isolated segments of nucleic acid molecules, e.g., by genetic engineering techniques such as those described in Sambrook et al. (ed.), Molecular Cloning: A Laboratory Manual, 3d ed., vol. 1-3, Cold Spring Harbor Laboratory Press, Cold Spring Harbor, N.Y., 2001. The term recombinant includes nucleic acid molecules that have been altered solely by addition, substitution, or deletion of a portion of the nucleic acid molecule. A recombinant or transformed organism or cell, such as a recombinant E. coli, is one that includes at least one exogenous nucleic acid molecule, such as a vector comprising a pre-LASSO probe (e.g., 16 of FIG. 1A).

Sample: Any biological, food, or environmental specimen (or source) that may contain (or is known to contain or is suspected of containing) a target nucleic acid molecule can be used in the methods herein.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. To compare two nucleic acid sequences, the options can be set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q -1 -r 2.

To compare two amino acid sequences, the options of Bl2seq can be set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (e.g., 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (i.e., 1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (i.e., 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least 75%, 80%, 85%, 90%, 95%, or 99% sequence identity.

Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least 60%, 70%, 80%, 90%, 95%, 98%, or 99% sequence identity determined by this method.

One of skill in the art will appreciate that these sequence identity ranges are provided for guidance only; it is possible that strongly significant homologs could be obtained that fall outside the ranges provided.

Subject: Living multi-cellular vertebrate organism, a category that includes human and non-human mammals, such as a veterinary subject (e.g., rabbit, rat, mouse, dog, cat, cow, pig, or non-human primate).

Transformed: A cell, such as a host cell, into which a nucleic acid molecule has been introduced, for example by molecular biology methods. Transformation encompasses all techniques by which a nucleic acid molecule might be introduced into a cell, including, but not limited to chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses. In one example, the transformed host cell is a bacterial cell, such as E. coli.

Vector: A nucleic acid molecule used to carry foreign genetic material, for example into a host cell, thereby producing a transformed or recombinant host cell. A vector may include nucleic acid sequences that permit it to replicate in the host cell, such as an origin of replication. A vector may also include a selectable marker gene, and other genetic elements. A vector can transduce, transform or infect a cell, thereby causing the cell to express nucleic acids and/or proteins. A vector optionally includes materials to aid in achieving entry of the nucleic acid into the cell, such as a viral particle, liposome, protein coating or the like. In one example, a vector is a plasmid, such as a plasmid exogenous to the cell or organism into which it is introduced. A vector can be linear (e.g., 14 of FIG. 1A) or circular (e.g., 16 of FIG. 1A).

### Overview

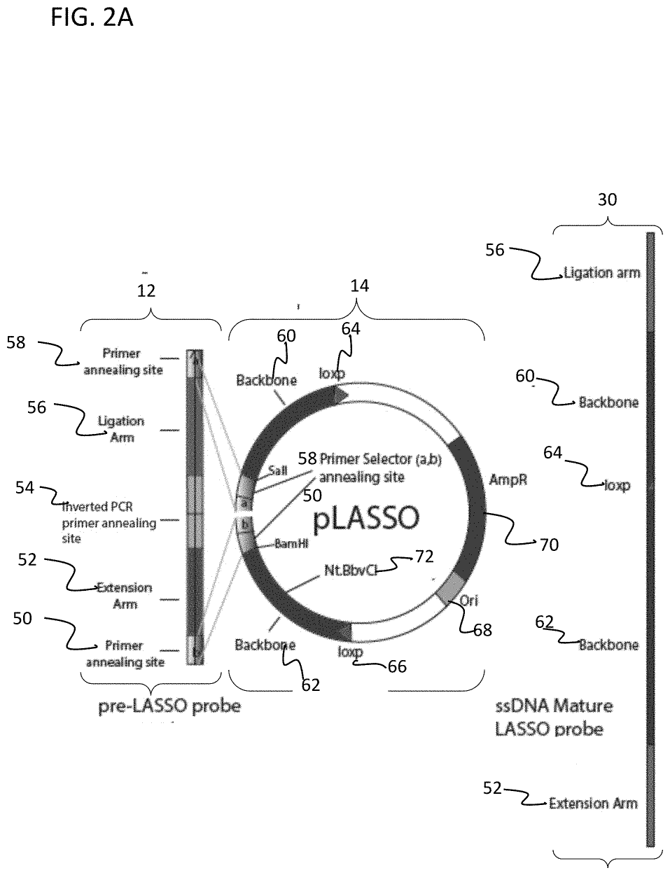

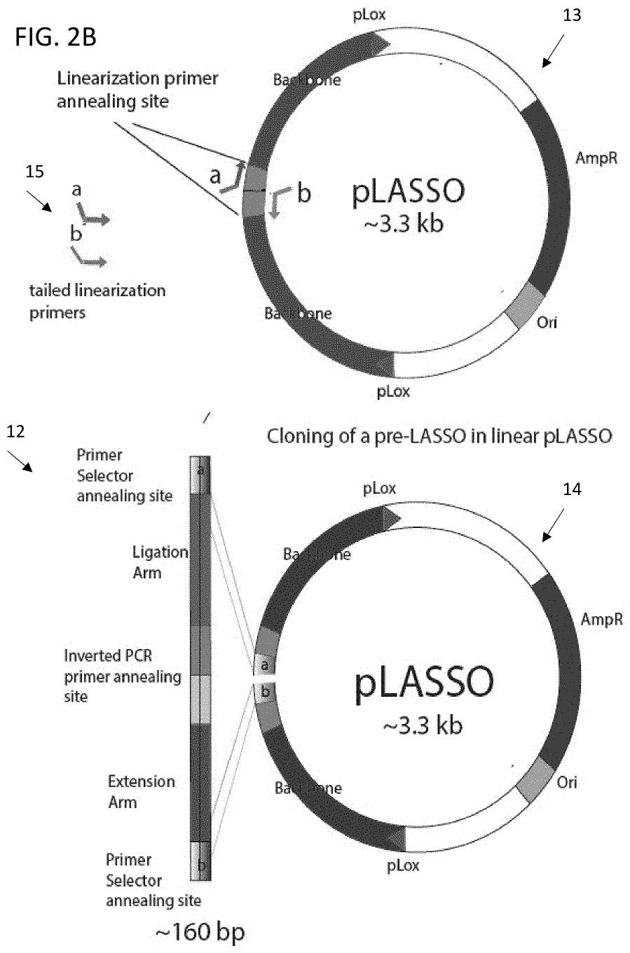

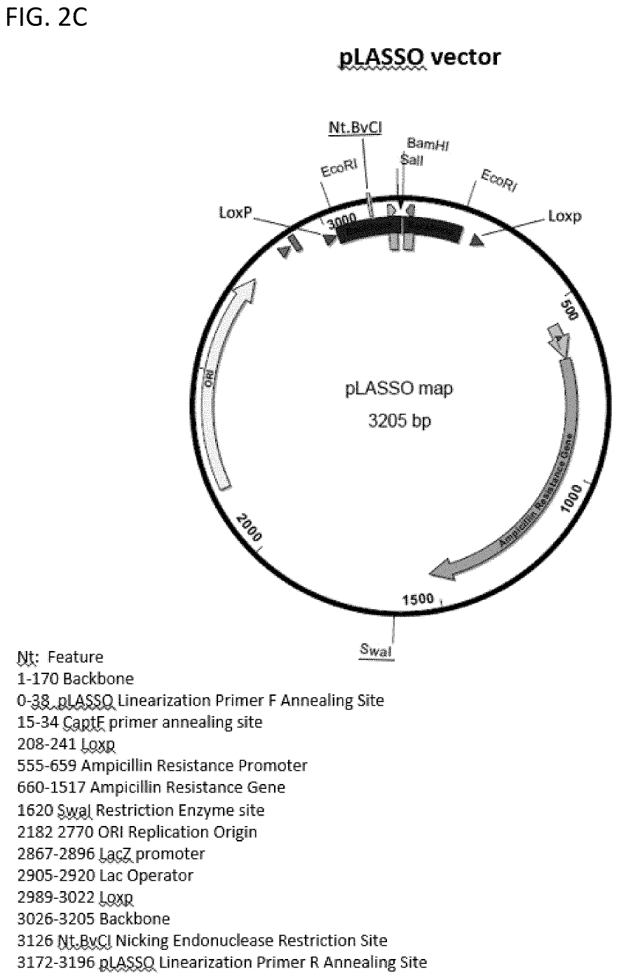

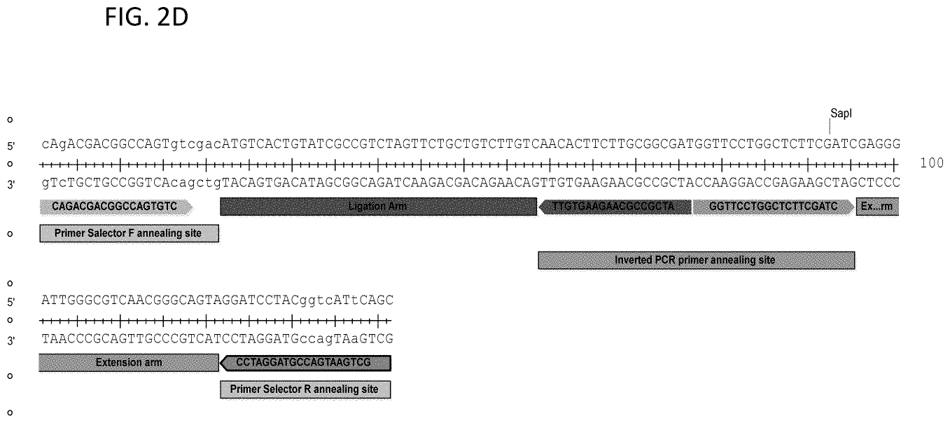

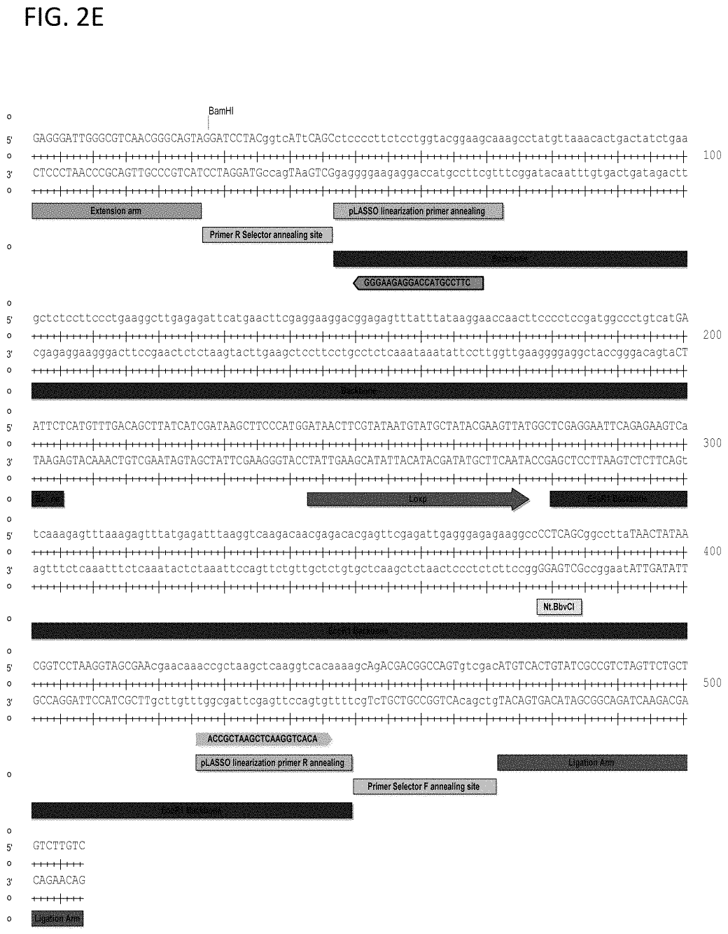

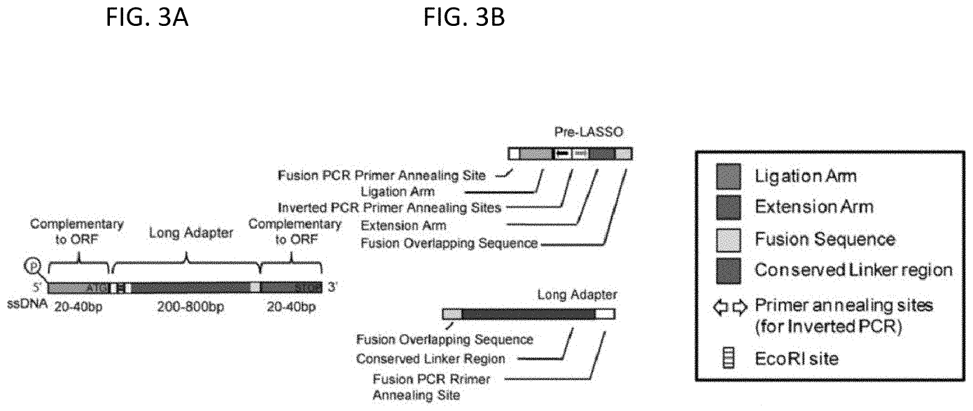

Provided herein is a DNA recombinase mediated assembly methods for generating mature ssDNA LASSO probes. As shown in FIGS. 1A and 2A-2D, the disclosed DNA recombinase mediated assembly methods 10 use a pre-LASSO probe 12 and vector pLASSO 14. In contrast, the previous long adapter based assembly procedure (FIGS. 1B, 3A, 3B) 100 uses a different pre-LASSO probe 110 and a long adapter sequence 112 instead of a vector 14. The resulting mature ssDNA LASSO probes can be used to produce genome-wide ORFeome libraries of prokaryotic and eukaryotic organisms, such as bacteria and humans (such as full length ORFs from human total cDNA). The resulting libraries can be used for next generating sequencing (NGS) analysis or shuttled in standard expression vectors for functional screening applications. Details can also be found in Tosi et al. (Biotechnol J., 17(2):e2100240, 2021), and Chkaiban et al. (Curr Protoc, (11):e278, 2021), herein incorporated by reference in their entireties.

In the prior method, (FIG. 1B), the resulting product did to produce a sufficiently pure population of mature ssDNA LASSO probes 128. Some of the mature ssDNA LASSO probes 128 had one arm (extension arm or ligation arm) that did not recognize or hybridize to the target nucleic acid molecule (e.g., hybridized to a non-target or non-specific region). In contrast, the new methods (FIG. 1A) provide mature ssDNA LASSO probes 30 that are purer than the prior method. For example, at least 40% of the mature ssDNA LASSO probes have extension and ligation arms that bind to the correct nucleic acid target (as compared to about 10% in the prior method). The disclosed methods omit the fusion PCR step, and instead use a recombination system.

As shown in FIGS. 1A and 2A-2B, 2D the pre-LASSO probe 12 is a synthetic oligonucleotide that includes a 5′-end and a 3′-end, each end containing a primer annealing site 50, 58. Following the 5′-end primer annealing site 50, the pre-LASSO probe 12 includes an extension arm 52, an inverted PCR primer annealing site 54, a ligation arm 56, and a 3′-end primer annealing site 58. In some examples, the pre-LASSO probe 12 is composed of naturally occurring nucleotides, non-naturally occurring nucleotides, or a mixture of both types. In some examples, the pre-LASSO probe 12 is at least 100 bp, at least 110 bp, at least 120 bp, at least 130 bp, at least 140 bp, at least 150 bp, or at least 160 bp, such as 100 to 500 bp, 100 to 400 bp, 100 to 300 bp, 100 to 200 bp, 100 to 170 bp, 100 to 160 bp, 140 to 180 bp, 140 to 170 bp, 150 to 170 bp, such as about 160 bp. The pre-LASSO probe 12 can be single stranded or double stranded DNA. In some examples, a ss pre-LASSO probe is converted to a ds DNA pre-LASSO probe for use in the disclosed methods. Exemplary ss pre-LASSO probes are provided in SEQ ID NOS: 1-3088, 3090-3093, 3117-3121, and 3126, and thus in some examples, a ss pre-LASSO probe is one including at least 80%, at least 85%, at least 90%, at least 95%, at least 98%, at least 99%, or 100% sequence identity to any of SEQ ID NOS: 1-3088, 3090-3093, 3117-3121, and 3126. In one example, a ss pre-LASSO probe includes at least 80%, at least 85%, at least 90%, at least 95%, at least 98%, at least 99%, or 100% sequence identity to SEQ ID NO: 3090, wherein N22 and N60 are the ligation and extension arms, respectively, having of a length of about 20-40 nt, 20-50 nt, or 20-80 nt each.

The primer annealing sites 50, 58 can specifically bind or hybridize to amplification primers (e.g., primers a and b 15 in FIG. 2b) used to amplify the pre-LASSO probe, and can include selector sequences for cloning. In some examples, the primer annealing sites do not form secondary or tertiary structures, such as hairpins. Each primer annealing site 50, 58 can be at least 10 base pairs (bp), such as at least 12, at least 15, or at least 20 bp, such as 10-50 bp, 10-40-bp, or 10-20 bp, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bp.

The sequence of the ligation arm 56 and extension arm 52 of the pre-LASSO probe 12 are complementary to the target sequence, and in the same 5′-3′ orientation of the target sequence to be captured. The sequence of the ligation arm 56 and extension arm 52 should only specifically hybridize to specifically bind to the target sequence, and not other sequences in the genome of the target organism. The ligation arm 56 and extension arm 52 end up as part of the ssDNA mature LASSO probe 30. The ligation arm 56 hybridizes or binds to a 5′-end of the target sequence, while the extension arm 52 hybridizes or binds to a 3′-end of the target sequence. For example, if the sequence of the target is 5′ATGCCAnnnnnnnTGATTGnnnnnn 3′ (SEQ ID NO: 3116) from the start (ATG) to the stop (TGA) codon, the ligation arm 56 and the extension arm 52 can have a sequence that begins with 5′ ATGCCAnnn and 5′TGATTGnnnnnn, respectively, and can be extended until the desired melting temperatures (Tm) are reached. In some examples, ligation arm 56 terminates in a C or G residue. In some examples the ligation arm 56 and extension arm 52 of the pre-LASSO probe 12 share 100% complementarity to a continuous 5′- and 3′-region, respectively, of target sequence. One skilled in the art will appreciate that lower complementarity is possible, such as at least 80%, at least 85%, at least 90%, at least 95%, at least 98%, or at least 99% complementarity to a continuous 5′- and 3′-region target sequence. The length of the ligation arm 56 and extension arm 52 can vary to achieve the desired Tm. In some examples, the Tm of extension arm 52 is about 50° C.-58° C., such as 52-56° C., such as 52° C., 53° C., or 54° C. In some examples, the Tm of ligation arm 56 is about 53° C.-61° C., such as 56-60° C., such as 57° C., 58° C., or 59° C. In some examples, the Tm of the extension arm 52 is 65-70° C. and ligation arm 56 is 70-75° C. In some examples, the Tm of the ligation arm 56 is about 2.5-5° C. (such as about 3, 4 or 5° C.) higher than the extension arm 52. In some examples, the Tm of the extension arm 52 and the ligation arm 56 are in the same range, such as 65-70° C. for both, or have the same Tm, such as 65° C. In some examples, each of ligation arm 56 and extension arm 52 is at least 10 bp, such as at least 12, at least 15, at least 20 bp, at least 25 bp, at least 30 bp, at least 40 bp, or at least 50 bp, such as 10-50 bp, 10-40-bp, 25-35 bp, or 20-40 bp, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bp.

In between the ligation arm 56 and extension arm 52 of the pre-LASSO probe 12 is an inverted PCR primer annealing site 54. The sequence of the inverted PCR primer annealing site 54 includes a restriction site that allows for asymmetric cutting (see steps F and G in FIG. 1A), such as a Type IIS restriction site that results in cleavage outside of its recognition sequence. Examples include BbvI, BcgI, BspMI, BspQ1, BtgZI, Esp3I, FokI, MboII, and SapI. In a specific example, a BspQ1 restriction site is present.

In some examples, an algorithm is used to design the sequence of the pre-LASSO probe 12. For example, thousands of ligation arm 56 and extension arm 52 sequences can be designed based on the target sequence, such as genomic or metagenomics DNA sequence(s). The algorithm can adjust the thresholds for target length, melting temperature, or the length of the ligation/extension arms 52, 56 to identify probe sequences. In one example, the algorithm first selects the ORF leading and trailing 32-mer sequences for the ligation arm 56 and extension arm 52, determining whether the last nucleotide of the arm is a cytosine or a guanine and that the melting temperature for the ligation arm 56 and extension arm 52 is 60° C.-85° C. and 55° C.-80° C., respectively. If one of these conditions are not satisfied, the algorithm increases the length of the arms by one nucleotide and the conditions re-tested until they are satisfied or the end of the ORF of the target sequences is reached.

In some examples, the target sequence captured is at least 1 Kb, at least 2 Kb, at least 3 Kb, at least 4 Kb, or at least 5 Kb, such as 1-6 Kb, 1-5 Kb, or 2-4 Kb.

In some examples, a pre-LASSO library is used, which is typically composed by thousands of different pre-LASSO probes 12. Such a library can be PCR amplified using primers that specifically hybridize or bind to primer annealing sites 50, 58. Different primer annealing sites 50, 58 within members of the pre-LASSO libraries can be used to selectively amplify sub-pools within the larger library. Exemplary pairs of primer annealing sites 50, 58 for pre-LASSO probe library amplification are provided in SEQ ID NOS: 1-3088.

As shown in FIGS. 2A-2C and 2E, vector pLASSO 14 is plasmid produced from the pLox2+ linear plasmid (New England Biolabs) and includes two backbone regions 60, 62 (e.g., nt 47-207 and 282-452 of FIG. 2E). As shown in FIG. 2B, the pLASSO plasmid can begin as a circular vector 13, and be linearized using PCR amplification with tailed primers a, b, 15. This results in a linear pLASSO plasmid 14. The pLASSO plasmid 14 provides two backbone regions 60, 62 for the ssDNA mature LASSO probe 30, and functional sites required for the assembly of the mature LASSO probe 30. The backbone regions 60, 62 have nucleic acid sequence that does not substantially hybridize or bind to a sequence within the target genome. In some examples, each backbone region 60, 62 includes a unique sequence tag that allows for subsequent isolation of all mature pLASSO probes containing the unique sequence. The length of the backbone regions 60, 62 can vary depending on the size of the target. In some examples, each backbone region 60, 62 is at least 100 bp, at least 150 bp, at least 200 bp, at least 250 bp, at least 300 bp, at least 350 bp, at least 400 bp, at least 500 bp, at least 460 bp, at least 700 bp, or at least 800 bp, such as 100 to 1000 bp, 100 to 800 bp, 200 to 800 bp, 200 to 400 bp, 400 to 800 bp, such as about 200, 400, or 800 bp. Vector pLASSO 14 includes two recombination sites 64, 66 (pink triangles in FIG. 1A, 2) (examples of recombination sites that can be use include loxP for Cre recombination, and for FRT for filppase (FLP) recombination), two selector primer annealing sites 50, 58 for linearization and specificity towards to a specific primer annealing site pre-LASSO probe, an origin of replication (Ori) 68, and a selectable marker 70, such as antibiotic resistance gene (e.g., ampicillin, hygromycin, chloroamphenicol, tetracycline, and kanamycin) to permit selection of appropriate colonies. The selector primer annealing sites 50, 58 are identical in sequence to the primer annealing sites 50, 58 in the pre-LASSO probe and are introduced into pLASSO 14 during PCR linearization with selector primers (see FIG. 2B, top, tailed linearization primers anneal to circular pLASSO at the light blue regions, resulting in linearization and addition of the primer annealing sites 50, 58 (a and b grey area in bottom panel FIG. 2B). Vector pLASSO 14 further includes a nicking endonuclease recognition site 72 (for example in the backbone), such as Nt.BbvCI, Nt.BstNBI, Nb.BtsI, or Nb.BsrDI. Vector pLASSO 14 can also include additional restriction enzyme sites such as SalI and BamHI (for example in or near each backbone 60, 62, which can be used for verification steps).

In some examples, the backbone sequence used includes at least 80%, at least 85%, at least 90%, at least 95%, at least 98%, at least 99%, or 100% sequence identity to any of SEQ ID NOS: 3089, 3122, and 3123.

As shown in FIGS. 1A and 2A, following the disclosed assembly process, the resulting ssDNA mature LASSO probe 30 generated using the method illustrated in FIG. 1A and described herein, includes from 5′ to 3′, extension arm 52, backbone 62, recombination site 64, backbone 60, and ligation arm 56. In some examples, ssDNA mature LASSO probe 30 is at least 300 nt, such as at least 400, at least 450, at least 500 nt, at least 550 nt, at least 600 nt, at least 650 nt, or at least 700 nt, such as 300-1000 nt, 300-800 nt, 300-700 bp, 400-700 nt, 500 to 700 nt, 550-650 nt, such as about 600 nt, about 625 nt, or about 650 nt.

An overview of the DNA recombinase mediated assembly method is shown in FIG. 1A (and contrasted with the prior long adapter based assembly method in FIG. 1B). As shown in step A of FIG. 1A, the pre-LASSO probe 12 is integrated into the linearized pLASSO vector 14, for example using sequence independent ligation (e.g., no restriction site is required). In some examples NEBbuilder or Gibson Assembly® reaction is used. In this reaction, a 5′ exonuclease generates long overhangs in the primer annealing sites 50, 58, allowing the primer annealing sites 50, 58 from the pre-LASSO probe 12 to anneal to the corresponding primer annealing sites 50, 58 of the linearized pLASSO vector 14. A polymerase (such as a DNA polymerase, such as one having low strand displacement, such as Kapa HiFi or Omni Klentaq LA) fills in the gaps of the annealed single strand regions, and a DNA ligase seals the nicks of the annealed and filled-in gaps. The pre-LASSO probe primer annealing sites 50 and 58 link with the primer selector annealing sequences 50, 58 of pLASSO (FIG. 1 A, a and b selectors) generating a circular pLASSO vector 16 containing the pre-LASSO probe 12 (step B of FIG. 1A). As shown in step C of FIG. 1A, the circular pLASSO vector 16 is introduced into host cells, such as bacterial cells (e.g., E. coli). Any method of transformation can be used, such as electroporation. In some examples, NEBuilder assembly solution is used for E. coli electroporation. The transformed cells can be grown in the presence of an appropriate selection growth media, depending on the selectable marker 70 in circular pLASSO vector 16 (e.g., ampicillin-containing media if the gene is AmpR). Resulting colonies that survive the selection media (such as growth in the presence of an appropriate antibiotic), are collected and the circular pLASSO vector 16 extracted/removed from the cells.

The circular pLASSO vector 16 can form supercoils, which can adversely affect recombination. Therefore, as shown in step D of FIG. 1A, the circular pLASSO vector 16 is subjected to treatment with a nicking endonuclease, which cleaves one of the two DNA strands of the circular pLASSO vector 16 by using a nicking endonuclease recognition site 72 located in backbone region 62 (see FIG. 2A) (nicking endonuclease recognition site 72 could be located elsewhere in circular pLASSO vector 16, such as in primer selector annealing site 50 or 58 from linearized pLASSO 14). The nicking endonuclease used will depend on the particular sequence of the nicking endonuclease recognition site 72. Treatment with the nicking endonuclease relaxes the supercoiled circular pLASSO vector 16. The relaxed/nicked form of the circular pLASSO vector 16 can improve subsequent DNA recombination.

Following treatment with a nicking endonuclease, the relaxed circular pLASSO vector 16 is treated with a recombinase (such as Cre- or FLP-exonuclease), the two recombination sites (e.g., pLox or FRT) sites 64, 66 in pLASSO recombine. This internal DNA recombination produces DNA minicircles 18 containing the pre-LASSO probe 12, and the remaining part of the pLASSO vector 20 (e.g., did not integrate the pre-LASSO probe 12) (can be about 2.7 kb) (step E of FIG. 1A). The process may also generate an approximately 6 kb double plasmid generated by inter-plasmid recombination. The minicircles containing a single pre-LASSO probe 18 are recovered by selective cutting with restriction enzyme and exonuclease digestion (e.g., exonuclease V) of the remaining part of the pLASSO vector 20 (step E of FIG. 1A). That is, the remaining part of the pLASSO vector 20 can be selectively removed or destroyed. For example, the remaining part of the pLASSO vector 20 can be incubated with one or more restriction enzymes that recognize a site not found in the minicircle 18. For example, as shown in FIG. 1A, step D and FIG. 2C, a SwaI site can be included. In some examples, a minicircle 18 is at least 300 bp, such as at least 400, at least 450, at least 500 bp, at least 550 bp, at least 600 bp, at least 650 bp, or at least 700 bp, such as 300-1000 bp, 300-800 bp, 300-700 bp, 400-700 bp, 500 to 700 bp, 550-650 bp, such as about 600 pb, about 625 bp, or about 650 bp.

The resulting minicircles 18 are subjected to inverse PCR (step F, FIG. 1A), resulting in a linearized minicircle 24 that includes extension arm 52 and ligation arm 56 flanking the backbone sequences 60, 62 (and 50% of recombination site 64 and 50% of recombination site 66). The primers used in the inverted PCR step include a first primer 17 that hybridizes to the inverted PCR primer annealing site 54 (from preLASSO), and includes a restriction enzyme site (e.g., a Type IIS (shifted cleavage) restriction enzyme, such as BspQI, BsaI, BsmBI, BbsI, Esp3I, BtgZI, BspMI, BsmFI, SapI restriction enzyme site, which recognize an asymmetric DNA sequence and cleaves outside its recognition site located in the inverted PCR primer annealing site 54 and cleaves the 3′-5′(bottom strand) a DNA strand exactly at the 5′ end of the extension arm. while the 5′-3′ DNA strand (top strand) is cut inside the “inverted PCR primer annealing site), and a second primer 19 that hybridizes to the inverted PCR primer annealing site 54 (from preLASSO) and its first three 5′ bases have phosphorothioate bonds and that protect this DNA strand from Lambda exonuclease digestion, and includes a 3′-uracil final base (e.g., exemplary second primer 19 sequence A*T*C*GCCGCAAGAAGTGTU3′ SEQ ID NO: 3105). The 3′-terminal uracil is used for subsequent primer removal using Uracil-DNA Glycosylase (USER enzyme). As shown in steps G-L of FIG. 1A, the linearized minicircle 24 is treated to remove the 5′- and 3′-end primer annealing sites (FIG. 2A, 54) are removed to generate the mature ssDNA LASSO probe 30. For example, linearized minicircle 24 is digested with a restriction enzyme that recognizes an asymmetric DNA sequence and cleaves outside its recognition site located in the “inverted PCR primer annealing site” and cleaves the 3′-5′ (bottom strand) a DNA strand exactly at the 5′ end of the extension arm, while the 5′-3′ DNA strand (top strand) is cut inside the “inverted PCR primer annealing site” (for example using BspQI) to produce 26, a lambda exonuclease (e.g., T7 Exonuclease) to remove/digest the top 5′-3′ DNA strand that is not protected by the 5′ phosphorothioate bonds, to produce 28, and USER enzyme to remove the inverted primer annealing site (54 of FIG. 2A), thereby generating the mature ssDNA LASSO probe 30, which can be used for capture experiments. For example, ssDNA LASSO probe 30 can be used for parallel DNA target capture by 5′-3′ gap filling after annealing to target sequences that flank the desired DNA fragments, and the massively parallel capture of fragments can be used for sequencing or expression experiments. In some examples, massively parallel capture is includes four phases: hybridization, capture, purification of circularized targets, and post capture PCR amplification. Such a reaction can be performed in a PCR thermal cycler. During the hybridization, the target nucleic acid (e.g., genomic DNA or cDNA) is incubated with one or more mature LASSO probes 30 (e.g., mature ssDNA LASSO probe library). The capture is performed by adding a Gap Filling Mix directly into the hybridization reaction, which contains a polymerase (such as DNA polymerase, such as one having low strand displacement, such as Kapa HiFi) and a thermostable ligase (such as a DNA ligase). The Gap Filling Mix (e.g., a thermostable DNA ligase, a DNA polymerase, dNTPs, glycerol, buffer, wherein the glycerol stabilizes the mix and allows storage at −20 C for several months. In some examples, the ligase is used at 0.025 U/ul to 0.3 U/ul, such as 0.25 U/ul. The gap that is in between ligation arm and extension arm hybridization sites is filled by the polymerase using free nucleotides and the ends of the probe are ligated by the ligase, resulting in a fully circularized loop containing the target nucleic acid sequence. The ssDNA circles, representing the LASSO probes containing target nucleic acid molecule(s) are isolated from the rest of the linear template dsDNA or the unreacted LASSO probes by incubation with one or more exonucleases. To enrich the captured target(s), PCR amplification can be performed using as a template the capture reaction that was subjected to exonuclease digestion and universal primers that anneal to a portion of the backbone sequence 60, 62. The capture can be verified by examination of the post capture PCR product on agarose gel to verify the presence of the expected size of the targeted nucleic acid regions. For NGS analysis, the post capture PCR product is purified and subjected to enzymatic fragmentation. NebNext Ultra (NEB) or other commercial kits can be used to prepare the fragmented library for NGS sequencing. For downstream expression experiments, the post capture PCR product can be subjected to a second round of PCR amplification using tailed primers containing Gateway attB1 (AttB1 CapF, GGGGACAAGTTTGTACAAAAAAGCAGGCTtcACCGCTAAGCTCAAGGTCACA SEQ ID NO: 3110) and attB2 (AttB2 CapR, GGGACCACTTTGTACAAGAAAGCTGGGTcctaatCTTCCGTACCAGGAGAAGG G SEQ ID NO: 3111) sequences. The purified PCR product is mixed with the Gateway ‘donor vectors’ (pDONR221) and the BP Clonase enzyme mix (Invitrogen). The purified BP reaction can be used for E. coli electroporation to generate an entry clone library for downstream expression.

In the previous long adapter based assembly method 100 (FIG. 1B, 3A-3B, also see WO 2016/197065), the pre-LASSO probe 110 (FIG. 3B top) is similar to pre-LASSO probe 12. However, the pre-LASSO probe 12 used in the disclosed DNA recombinase mediated assembly method has different terminal sequence regions, with different functions. However, the central inverted PCR primer annealing site 54 is the same for both assembly methodologies. The long adapter sequence 112 (FIG. 1B, FIG. 3B, bottom) is a linear dsDNA sequence of ˜200-800 bp, but it is not a plasmid and does not contain any recombination sites. In the previous long adapter based assembly method 100, the long adapter 112 is attached to the pre-LASSO probe 110 via fusion PCR using primers that anneal in one end of the long adapter and one end of the pre-LASSO probe (step b, FIG. 1B). The fusion product 114 is subsequently digested with EcoR1 restriction enzyme that produces a fusion product with sticky ends 116 (step c, FIG. 1B). The fusion product with sticky ends 116 is circularized when T4 Ligase is added (step d, FIG. 1B), generating a DNA minicircle 118. Thus, both the disclosed DNA recombinase mediated assembly methods 10 and the previous long adapter based assembly methods 100 produce DNA minicircles 18 and 118. From this point, the assembly steps for both assembly approaches 10 and 100 are identical. As shown in FIG. 1B, the minicircles 118 are subjected to inverted PCR so that the annealing arms are made to flank the backbone sequence in the final configuration, the resulting linearized minicircle 120 subjected to BspQ1 digestion, lambda exonuclease digestion, and USER digestion, resulting in a mature ssDNA LASSO probe 128, which can be used for capture experiments. Mature ssDNA LASSO probe 128 differs from mature ssDNA LASSO probe 30 in that mature ssDNA LASSO probe 128 does not include a recombination site (e.g., no loxp site).

It is also shown herein that the target capture process efficiency can be increased by increasing the ligase concentration, for example by at least 2-fold, at least 3-fold, at least 5-fold, or at least 10-fold over prior methods (such as at least 10-fold, such as 0.25 U/μl). In some examples, a DNA polymerase with low strand displacement is used, such as Kapa HiFi polymerase, for example to capture targets up to about 5 Kb (such as 1 to 6 Kb, such as 1-5.5 Kb, 1-5 Kb, 1-4.5 Kb, 1-4 Kb, 1-2 Kb, or 1-3 Kb). It is also shown herein that when the melting temperature (Tₘ) of the extension arm and ligation arm are in the same range of 65-70° C., a greater percentage were able to capture homogeneously (MLD of 0.77) 96.26% of the targeted ORFs. In addition, these conditions resulted in a 315.69 fold enrichment of coverage for captured target versus coverage for captured non targeted ORFs. Thus, in some examples, the melting temperature (Tₘ) of the extension arm and ligation arm in the compositions and methods herein are in the same range of 65-70° C.

### Mature LASSO Probes

Provided herein are new single stranded (ss) DNA Long Adapter Single Stranded Oligonucleotide (LASSO) probes. Such probes include, from 5′ to 3′, (1) a ligation arm sequence complementary to a 5′ region of a target sequence, (2) a backbone sequence that is not complementary to the target sequence, and includes a recombination site (e.g., loxp, frt), and (3) an extension arm sequence at least 20 nt complementary to a 3′ region of the target sequence, wherein the ligation arm sequence and extension arm sequence are complementary to 5′ and 3′ regions of a single target sequence, respectively. In some examples, and the complementary regions a single target sequence are at least 100 nt apart, such as at least 200 nt, at least 300 nt, at least 400 nt, at least 500 nt, at least 600 nt, at least 700 nt, at least 800 nt, at least 1000 nt, at least 5000 nt, at least 10,000 nt, at least 20,000 nt, at least 30,000 nt, at least 50,000 nt, or at least 100,000 nt apart, such as 200-30,000 nt 100-500, 100-1000, 100-5,000, 100-10,000, 100-20,000, or 100-30,000 nt apart on the target sequence.

In some examples, the ligation arm sequence is at least 20 nt, at least 25 nt, at least 30 nt, or at least 40 nt, such as 20-40 nt. In some examples, the backbone sequence is at least 100 nt, at least 150 nt, at least 200 nt, at least 300 nt, at least 350 nt, at least 400 nt, at least 500 nt, at least 600 nt, at least 700 nt, at least 800 nt, or at least 1000 nt, such as 100-2500, 200-500, 200-2000, 200-2500, 200-1500, 200-1000, 200-800, 200-400 nt, 250-350 nt, 300-400 nt, or 250-300 nt. In some examples, the extension arm sequence is at least 20 nt, at least 25 nt, at least 30 nt, or at least 40 nt, such as 20-40 nt. In some examples, combinations of such lengths are used. In some examples, the ssDNA LASSO probe is at least 200 nt, at least 400 nt, at least 500 nt, at least 600 nt, at least 650 nt, at least 700 nt, or at least 800 nt, such as about 200 to 800 nt, 400 to 800 nt, or 500-700 nt.

In some examples, the target sequence is a DNA sequence, such as a coding or noncoding DNA sequence, for example cDNA or genomic DNA. In some examples, the target sequence is an RNA sequence, such as mRNA or miRNA sequence. In some examples, the target sequence is a complete or partial open reading frame, complete or partial intronic DNA regions, or a noncoding sequence such as lincRNA or regulatory RNA. In some examples, the target sequence is a prokaryotic nucleic acid sequence, such as a bacterial nucleic acid sequence. In some examples, the target sequence is a eukaryotic nucleic acid sequence, such as a mammalian nucleic acid sequence, fungal nucleic acid sequence, or a plant nucleic acid sequence, such as a human nucleic acid sequence. In some examples, the target sequence is a viral nucleic acid sequence. In some examples, the target sequence is a single contiguous target sequence, such as a genomic sequence, lncRNA, mRNA, or cDNA.

### Methods of Making ssDNA LASSO Probes

Provided herein are methods of generating the ssDNA LASSO probes described herein. Such methods utilize a double stranded pre-LASSO probe (e.g., see 12 in FIG. 1A) (such as one that is about 80-200 base pairs (bp) long, such as about 160 bp), having from 5′ to 3′(i) a first primer annealing site sequence, (ii) the extension arm sequence, (iii) an inverted PCR primer annealing site comprising a restriction site that allows for asymmetric cutting, (iv) the ligation arm sequence, and (v) a second primer annealing site sequence. In some examples, all or a subset of the pre-LASSO probes have the same primer annealing sequences. The methods also utilize a double stranded linear pLASSO vector (e.g., see 14 in FIG. 1A), having from 5′ to 3′ (e.g., “a” in 14 of FIG. 2A is the 5′ end) (i) the second primer annealing site sequence (i.e., the second primer annealing site sequence of the pre-LASSO probe), (ii) a first backbone region that does not substantially hybridize to the target sequence, (iii) a first recombination site, (iv) a selectable marker, (v) an origin of replication, (vi) a second recombination site, (vii) a second backbone region that does not substantially hybridize to the target sequence and (viii) the first primer annealing site sequence (i.e., the first primer annealing site sequence of the pre-LASSO probe), wherein the double stranded linear pLASSO vector further includes a nicking endonuclease recognition site (for example in the backbone), a restriction site not in the backbone (for example between the first recombination site and the selectable marker) used to digest a plasmid (e.g., SwaI), and optionally a first restriction endonuclease site (such as SalI) and a second restriction endonuclease site (such as BamHI).

The methods include contacting the ds DNA pre-LASSO probe with the ds linear pLASSO vector using sequence independent ligation conditions described above for step A in FIG. 1A, thereby generating a circular pLASSO vector containing the pre-LASSO probe (e.g., see 16 in FIG. 1A). The resulting circular pLASSO vector is introduced (e.g., transformed) into host cells, thereby generating transformed host cells comprising the circular pLASSO vector. The resulting transformed cells are grown in the presence of a growth media (such as solid or liquid media) containing reagents that do not permit growth of the host cells in the absence of the selectable marker (e.g., if the circular pLASSO vector contains an AmpR gene, the cells will grow in ampicillin media). The circular pLASSO vector is subsequently extracted or removed from the transformed host cells, and then contacted or incubated with a nicking endonuclease specific for the nicking endonuclease recognition site, under conditions that cleave one nucleic acid strand of the extracted circular pLASSO vector, thereby producing a relaxed circular pLASSO vector. The relaxed circular pLASSO vector is contacted or incubated with a recombinase specific for the first and second recombination sites (such as Cre or Flp), under conditions that recombination of the relaxed circular pLASSO vector occurs, thereby generating (i) a plasmid comprising a recombination site, the selection marker, and the origin of replication and (ii) a minicircle comprising the double stranded pre-LASSO probe, the first and second backbones, and a recombination site. The plasmid is digested with a restriction enzyme (the one used will be based on the restriction site in pLASSO 16 not in either backbone, such as SwaI (or other restriction enzyme) and exonuclease V. The minicircle is subjected to inverse PCR, using a first primer and a second primer that hybridize to the inverted PCR primer annealing site, wherein the first primer includes a restriction enzyme site (e.g., a Type IIS (shifted cleavage) restriction enzyme, such as BspQI, BsaI, BsmBI, BbsI, Esp3I, BtgZI, BspMI, BsmFI, SapI restriction enzyme site, which recognize an asymmetric DNA sequence and cleaves outside its recognition site located in the inverted PCR primer annealing site 54 and cleaves the 3′-5′(bottom strand) a DNA strand exactly at the 5′ end of the extension arm. while the 5′-3′ DNA strand (top strand) is cut inside the “inverted PCR primer annealing site) and wherein the second primer comprises a 3′-uracil and the first three 5′-end nt are modified nucleotides resistant to exonuclease treatment (e.g., connected by phosphorothioate bonds that are resistant to lambda exonuclease treatment), thereby generating a linear double stranded minicircle with a 5′ end and 3′ end, wherein the 5′ end of the linear double stranded minicircle is the first primer annealing site at the 3′ end of the linear double stranded minicircle is the second primer annealing site. The linear double stranded minicircle is subjected to conditions that (1) remove all or part of the first and second primer annealing sites from the 5′ and 3′ end of the linear double stranded minicircle by restriction digestion and/or glycosylase digestion and (2) remove one of the two strands of the digested linear double stranded minicircle, thereby producing the ssDNA LASSO.

In some examples, removing all or part of the first and second primer annealing sites from the 5′ and 3′ end of the linear double stranded minicircle includes removing all or part of the first and second primer annealing sites from the 5′ and 3′ end of the linear double stranded minicircle by restriction digestion and/or glycosylase digestion to produce a digested linear double stranded minicircle, and removing one of the two strands of the digested linear double stranded minicircle, thereby producing the ssDNA LASSO probe.

In some examples, removing one of the two strands of the digested linear double stranded minicircle includes using a lambda exonuclease.

In some examples, the double stranded pre-LASSO probe includes a plurality of double stranded pre-LASSO probes, and the method creates a library of ssDNA LASSOs that can target a plurality of nucleic acid sequences, such as at least 2, at least 10, at least 50, at least 100, at least 200, at least 1000, at least 10,000 at least, or at least 100,000 o different nucleic acid target sequences, for example in the same sample.

### Methods of Using ssDNA LASSO Probes

Also provided are methods of using the ssDNA LASSO probes generated using the disclosed methods. In some examples, the method includes using the ssDNA LASSO probes to detecting one or more target sequences. For example, such a method can include contacting a sample containing one or more target sequences with one or more ssDNA LASSO probes provided herein, wherein the ligation arm sequence and the extension arm sequence are complimentary to a 5′ region of the target sequence and to a 3′ region of the target sequence, respectively. The ligation arm sequence and extension arm sequence are allowed to hybridize to the target sequence. Gap filling is used to copy the target sequence between the ligation arm sequence and extension arm sequence using a polymerase (such as a DNA polymerase, such as one with low strand displacement, such as Kapa HiFi polymerase), thereby generating a ssDNA circle containing a copy the targeted DNA sequence. The resulting molecule is ligated, thereby generating a circular single stranded DNA fragment comprising the target sequence. The circular single-stranded DNA fragment comprising the target sequence is isolated, for example by digesting linear DNA in the sample (e.g., by adding directly to the capture reaction an aliquot of “linear DNA digestion solution” containing Exonuclease I, Exonuclease III and Lambda Exonuclease). The circular single stranded DNA fragment comprising the target sequences can then be amplified, for example using PCR, thereby detecting the target sequences (for example by detecting expected size DNA target sequence amplicons, e.g., using gel electrophoresis or the Bioanalizer).

In some examples, method detects a plurality of different target sequences, and the method includes contacting the sample comprising the target sequences with a plurality of ssDNA LASSOs, wherein the plurality of ssDNA LASSOs comprise sequences complementary to the different target sequences, such as at least 2, at least 10, at least 50, at least 100, at least 200, at least 1000, at least 10,000 at least, or at least 100,000 different nucleic acid target sequences, for example in the same sample.

In some examples, the target sequences are at least 200 nt long, such as at least 500, at least 1000, at least 5,000, at least 10,000, at least 20,000, at least 30,000, at least 50,000, at least 100,000, at least 500,000, at least 1,000,000 or more nt. In some examples, the hybridizing and the gap filling are performed at 55-75° C., such as 65° C.

In some examples, the sample includes eukaryotic or prokaryotic genomic DNA (gDNA), such as human gDNA. In one example, a sample includes mitochondrial DNA. Exemplary samples that can be used, include stool, tissue lysate, cell lysate, sputum, blood serum/plasma, bone marrow, saliva, and a tissue swab.

Also provided are libraries of target sequences generated by the disclosed methods.

The mature ssDNA LASSO probes provided herein can be used to target full-length open reading frames (ORFs) and genomic DNA, such as 100s or 1000s thousands full length ORF in a pooled format. In some examples, the target nucleic acid molecule is at least 1 kb, at least 2 kb, at least 3 kb, at least 4 kb, at least 5 kb, or more.

### Exemplary Target Nucleic Acid Molecules

In some examples the methods disclosed herein are used to detect a target nucleic acid molecule such DNA or RNA (such as cDNA, genomic DNA, mRNA, miRNA, etc.) in a eukaryote or prokaryote. Thus, in some examples, the extension and ligation arms of a pre-LASSO probe or mature LASSO probe have sufficient complementarity to hybridize to a target nucleic acid molecule (such as having at least 80%, at least 90%, at least 95%, at least 99%, or 100% sequence complementarity to the target) from a eukaryote or prokaryote, such as a pathogen or mammalian cells, such as a target nucleic acid molecule associate with a disease. For example, pathogens can have conserved DNA or RNA sequences specific to that pathogen (for example conserved sequences are known in the art for HIV, bird flu and swine flu), and cells may have specific DNA or RNA sequences unique to that cell. In some examples, a target nucleic acid molecule is associated with a disease or condition.

In specific non-limiting examples, the target nucleic acid sequence is associated with a tumor (for example, a cancer). Numerous chromosome abnormalities (including translocations and other rearrangements, reduplication (amplification) or deletion) have been identified in neoplastic cells, especially in cancer cells, such as B cell and T cell leukemias, lymphomas, breast cancer, ovarian cancer, colon cancer, neurological cancers and the like.

Exemplary target nucleic acids include, but are not limited to: the SYT gene located in the breakpoint region of chromosome 18q11.2 (common among synovial sarcoma soft tissue tumors); HER2, also known as c-erbB2 or HER2/neu (a representative human HER2 genomic sequence is provided at GENBANK® Accession No. NC_000017, nucleotides 35097919-35138441) (HER2 is amplified in human breast, ovarian, gastric, and other cancers); p16 (including D9S1749, D9S1747, p16(INK4A), p14(ARF), D9S1748, p15(INK4B), and D9S1752) (deleted in certain bladder cancers); EGFR (7p12; e.g., GENBANK® Accession No. NC_000007, nucleotides 55054219-55242525), MET (7q31; e.g., GENBANK® Accession No. NC_000007, nucleotides 116099695-116225676), C-MYC (8q24.21; e.g., GENBANK® Accession No. NC_000008, nucleotides 128817498-128822856), IGF1R (15q26.3; e.g., GENBANK® Accession No. NC_000015, nucleotides 97010284-97325282), D5S271 (5p15.2), KRAS (12p12.1; e.g. GENBANK® Accession No. NC_000012, complement, nucleotides 25249447-25295121), TYMS (18p11.32; e.g., GENBANK™ Accession No. NC_000018, nucleotides 647651-663492), CDK4 (12q14; e.g., GENBANK® Accession No. NC_000012, nucleotides 58142003-58146164, complement), CCND1 (11q13, GENBANK® Accession No. NC_000011, nucleotides 69455873-69469242), MYB (6q22-q23, GENBANK® Accession No. NC_000006, nucleotides 135502453-135540311), lipoprotein lipase (LPL) (8p22; e.g., GENBANK® Accession No. NC_000008, nucleotides 19840862-19869050), RB1 (13q14; e.g., GENBANK® Accession No. NC_000013, nucleotides 47775884-47954027), p53 (17p13.1; e.g., GENBANK® Accession No. NC_000017, complement, nucleotides 7512445-7531642), N-MYC (2p24; e.g., GENBANK® Accession No. NC_000002, complement, nucleotides 15998134-16004580), CHOP (12q13; e.g., GENBANK® Accession No. NC_000012, complement, nucleotides 56196638-56200567), FUS (16p11.2; e.g., GENBANK® Accession No. NC_000016, nucleotides 31098954-31110601), FKHR (13p14; e.g., GENBANK® Accession No. NC_000013, complement, nucleotides 40027817-40138734), aALK (2p23; e.g., GENBANK® Accession No. NC_000002, complement, nucleotides 29269144-29997936), Ig heavy chain, CCND1 (11913; e.g., GENBANK® Accession No. NC_000011, nucleotides 69165054-69178423), BCL2 (18q21.3; e.g., GENBANK® Accession No. NC_000018, complement, nucleotides 58941559-59137593), BCL6 (3q27; e.g., GENBANK® Accession No. NC_000003, complement, nucleotides 188921859-188946169), AP1 (1p32-p31; e.g., GENBANK® Accession No. NC_000001, complement, nucleotides 59019051-59022373), TOP2A (17q21-q22; e.g., GENBANK® Accession No. NC_000017, complement, nucleotides 35798321-35827695), TMPRSS (21q22.3; e.g., GENBANK® Accession No. NC_000021, complement, nucleotides 41758351-41801948), ERG (21q22.3; e.g., GENBANK® Accession No. NC_000021, complement, nucleotides 38675671-38955488); ETV1 (7p21.3; e.g., GENBANK® Accession No. NC_000007, complement, nucleotides 13897379-13995289), EWS (22q12.2; e.g., GENBANK™ Accession No. NC_000022, nucleotides 27994017-28026515); FLI1 (11q24.1-q24.3; e.g., GENBANK® Accession No. NC_000011, nucleotides 128069199-128187521), PAX3 (2q35-q37; e.g., GENBANK® Accession No. NC_000002, complement, nucleotides 222772851-222871944), PAX7 (1p36.2-p36.12; e.g., GENBANK® Accession No. NC_000001, nucleotides 18830087-18935219), PTEN (10q23.3; e.g., GENBANK® Accession No. NC_000010, nucleotides 89613175-89718512), AKT2 (19q13.1-q13.2; e.g., GENBANK® Accession No. NC_000019, complement, nucleotides 45428064-45483105), MYCL1 (1p34.2; e.g., GENBANK™ Accession No. NC_000001, complement, nucleotides 40133685-40140274), REL (2p13-p12; e.g., GENBANK® Accession No. NC_000002, nucleotides 60962256-61003682) and CSF1R (5q33-q35; e.g., GENBANK® Accession No. NC_000005, complement, nucleotides 149413051-149473128).

### Exemplary Pathogen/Microbe Nucleic Acid Molecule Targets

In some examples the methods disclosed herein are used to detect a nucleic acid molecule from a pathogen. Thus, in some examples, the extension and ligation arms of a pre-LASSO probe or mature LASSO probe are complementary to a target nucleic acid molecule from a pathogen. Any pathogen or microbe nucleic acid molecule can be detected using the methods and molecules provided herein. A non-limiting list of pathogens having nucleic acid molecules that can be detected using the methods and molecules provided herein are provided below.

For example, target nucleic acid molecule can be from a virus, such as positive-strand RNA viruses and negative-strand RNA viruses. Exemplary target positive-strand RNA viruses include, but are not limited to: Picornaviruses (such as Aphthoviridae [for example foot-and-mouth-disease virus (FMDV)]), Cardioviridae; Enteroviridae (such as Coxsackie viruses, Echoviruses, Enteroviruses, and Polioviruses); Rhinoviridae (Rhinoviruses)); Hepataviridae (Hepatitis A viruses); Togaviruses (examples of which include rubella; alphaviruses (such as Western equine encephalitis virus, Eastern equine encephalitis virus, and Venezuelan equine encephalitis virus)); Flaviviruses (examples of which include Dengue virus, West Nile virus, and Japanese encephalitis virus); Calciviridae (which includes Norovirus and Sapovirus); and Coronaviruses (examples of which include SARS coronaviruses, such as the Urbani strain, and SARS-CoV-2). Exemplary negative-strand RNA viruses include, but are not limited to: Orthomyxyoviruses (such as the influenza virus), Rhabdoviruses (such as Rabies virus), and Paramyxoviruses (examples of which include measles virus, respiratory syncytial virus, and parainfluenza viruses).

Viruses also include DNA viruses. DNA viruses include, but are not limited to: Herpesviruses (such as Varicella-zoster virus, for example the Oka strain; cytomegalovirus; and Herpes simplex virus (HSV) types 1 and 2), Adenoviruses (such as Adenovirus type 1 and Adenovirus type 41), Poxviruses (such as Vaccinia virus), and Parvoviruses (such as Parvovirus B19).

Another group of viruses includes Retroviruses. Examples of retroviruses include, but are not limited to: human immunodeficiency virus type 1 (HIV-1), such as subtype C; HIV-2; equine infectious anemia virus; feline immunodeficiency virus (FIV); feline leukemia viruses (FeLV); simian immunodeficiency virus (SIV); and avian sarcoma virus.

In one example, a target nucleic acid molecule is from one or more of the following: HIV-1; Hepatitis A virus; Hepatitis B (HB) virus; Hepatitis C (HC) virus; Hepatitis D (HD) virus; a respiratory virus (such as influenza A & B, respiratory syncytial virus, human parainfluenza virus, human metapneumovirus, severe acute respiratory syndrome coronavirus (SARS-CoV-1), or SARS-CoV-2), or West Nile Virus.

Pathogens also include bacteria. Bacteria can be classified as gram-negative or gram-positive. Exemplary target gram-negative bacteria include, but are not limited to: Escherichia coli (e.g., K-12 and O157:H7), Shigella dysenteriae, and Vibrio cholerae. Exemplary target gram-positive bacteria include, but are not limited to: Bacillus anthracis, Staphylococcus aureus, Listeria, pneumococcus, gonococcus, and streptococcal meningitis. In one example, a target nucleic acid molecule is from one or more of Group A Streptococcus; Group B Streptococcus; Helicobacter pylori; Methicillin-resistant Staphylococcus aureus; vancomycin-resistant enterococci; Clostridium difficile; E. coli (e.g., Shiga toxin producing strains); Listeria; Salmonella; Campylobacter; B. anthracis (such as spores); Chlamydia trachomatis; Ebola, or Neisseria gonorrhoeae.

Protozoa, nemotodes, and fungi are also types of pathogens. In some examples, a target nucleic acid molecule is from one or more of Plasmodium (e.g., Plasmodium falciparum to diagnose malaria), Leishmania, Acanthamoeba, Giardia, Entamoeba, Cryptosporidium, Isospora, Balantidium, Trichomonas, Trypanosoma (e.g., Trypanosoma brucei), Naegleria, or Toxoplasma. In some examples, a target nucleic acid molecule is from one or more of Coccidiodes immitis or Blastomyces dermatitidis.

### Exemplary Samples

Any biological, food, or environmental specimen that may contain (or is known to contain or is suspected of containing) a target nucleic acid molecule can be used in the methods herein. Samples can also include fermentation fluid, reaction fluids (such as those used to produce desired compounds, such as a pharmaceutical agents), and tissue or organ culture fluid.

Biological samples are usually obtained from a subject and can include genomic DNA, RNA (including mRNA), protein, cells, or combinations thereof. Examples include a tissue or tumor biopsy, fine needle aspirate, bronchoalveolar lavage, pleural fluid, spinal fluid, saliva, sputum, surgical specimen, lymph node fluid, ascites fluid, peripheral blood (such as serum or plasma), bone marrow, urine, semen, buccal swab, and autopsy material. Techniques for acquisition of such samples are known in the art (for example see Schluger et al. J. Exp. Med. 176:1327-33, 1992, for the collection of serum samples). Serum or other blood fractions can be prepared in the conventional manner. Thus, using the methods provided herein, target nucleic acid molecule in the body can be detected.

Environmental samples include those obtained from an environmental media, such as water, air, soil, dust, wood, plants, or food (such as a swab of such a sample). In one example, the sample is a swab obtained from a surface, such as a surface found in a building or home. Thus, using the methods provided herein, microbes found in the environment can be detected, such as a pathogen.

In one example the sample is a food sample, such as a meat, dairy, fruit, or vegetable sample. For example, using the methods provided herein, adulterants in food products can be detected, such as a pathogen or toxin. For example, beverages (such as milk, cream, soda, bottled water, flavored water, juice, and the like), and other liquid or semi-liquid products (such as yogurt) can be analyzed with the methods provided herein.

In one example the sample is a sample from a chemical reaction, such as one used to produce desired compounds, such as a pharmaceutical agent, such as a biologic.

In other examples, a sample includes a control sample, such as a sample known to contain, or not contain, a particular amount of the target nucleic acid molecule.

Once a sample has been obtained, the sample can be used directly, concentrated (for example by centrifugation or filtration), purified, liquefied, diluted in a fluid, or combinations thereof. In some examples, proteins, cells, nucleic acids, or pathogens are extracted from the sample, and the resulting preparation (such as one that includes isolated cells, pathogens, DNA, or RNA) analyzed using the methods provided herein.

### Compositions and Kits

Also provided are compositions that include one or more of the disclosed ssDNA LASSO probes, such as those that include a pharmaceutically acceptable carrier (e.g., water, saline). In some examples, a composition includes a plurality of ssDNA LASSO probes, having ligation and extension arm sequences complementary to at least 2, at least 3, at least 4, at least 5, at least 10, at least 50, at least 100, at least 500, at least at least 1000, at least 10,000, at least 100,000, or at least at least 100,000,000 different target sequences. Such compositions can be in a container, such as a glass or plastic container, wherein the composition is liquid, frozen, or freeze-dried.

Also provided are kits that include one or more of the disclosed ssDNA LASSO probes (or compositions). Such kits can include other elements, such as one or more endonucleases, one or more exonucleases, one or more polymerases (such as a DNA polymerase, such as one with low strand displacement, such as Kapa HiFi polymerase), one or more ligases, one or more recombinases; one or more reagents for PCR, or combinations thereof. In specific examples, the kit includes one or more of the disclosed ssDNA LASSOs probes (such as a probe library), and one or more of a gap filling mix (e.g., a thermostable DNA ligase, a DNA polymerase [such as one with low strand displacement, such as Kapa HiFi polymerase]), dNTPs, glycerol, buffer), linear DNA digestion solution (e.g., Exonucleases I, III and Lambda, buffer and glycerol), oligonucleotide primers for post capture PCR reaction, post capture PCR master mix (e.g., DNA polymerase, dNTPs and buffer), and a positive control for the capture reaction (e.g., a LASSO probe that captures 1 kb target sequence within the genome of the phage M13mp18 single stranded DNA, or the LASSO probe and an aliquot of M13mp18 single stranded DNA (New England Biolab N4040S)). In some examples, the elements of the kit are in separate containers.

### Example 1

### Pre-LASSO Probe Amplification

This example describes methods that can be used to amplify a pre-LASSO probe (e.g., 12 in FIG. 1A, 2A-2B, 2D).

A stock solution of pre-LASSO probe Oligo Pool is prepped by re-suspending in 10 mM Tris buffer, pH 8.0 to a concentration of at least 20 ng/μL. Stock solution concentration (ng/μL)=Total yield (ng)/resuspension volume (μL). The KAPA HiFi HotStart PCR Kit can be used to perform PCR using the pre-LASSO primer pair with the primer annealing site of the pre-LASSO library. If the pre-LASSO library is composed of different sub-libraries, use the appropriate pre-LASSO primers pairs to select the sub-library of choice.

The PCR reaction is as follows:

| | FINAL | PER 25 μL |
|---|---|---|
| COMPONENT | CONCENTRATION | REACTION |
| 5× KAPA HiFi Fidelity | 1× | 5.0 μL |
| Buffer | | |
| 10 mM each dNTP Mix | 0.3 mM each dNTP | 0.75 μL |
| 10 μM Forward Primer | 0.3 μM | 0.75 μL |
| 10 μM Reverse primer | 0.3 μM | 0.75 μL |
| Twist Oligo Pool (20 ng/μL) | 0.4 ng/μL | 0.5 μL |
| KAPA HiFi HotStart DNA | 0.5 U/reaction | 0.5 μL |
| Polymerase (1 U/μL) | | |
| PCR grade water | — | Fill to 25 μL |

PCR Reaction Conditions

| CYCLING | | | |
|---|---|---|---|
| STEP | TEMPERATURE | | DURATION |
| 1 | Initialization | 3 min at 95° C. | 1× |
| | Denaturation | | |
| 2 | Denaturation | 20 sec at 98° C. | 6-12 Cycles** |
| 3 | Annealing | 15 sec at 58° C. | |
| 4 | Extension | 15 sec at 72° C. | |
| 5 | Final Extension | 1 min at 72° C. | 1× |

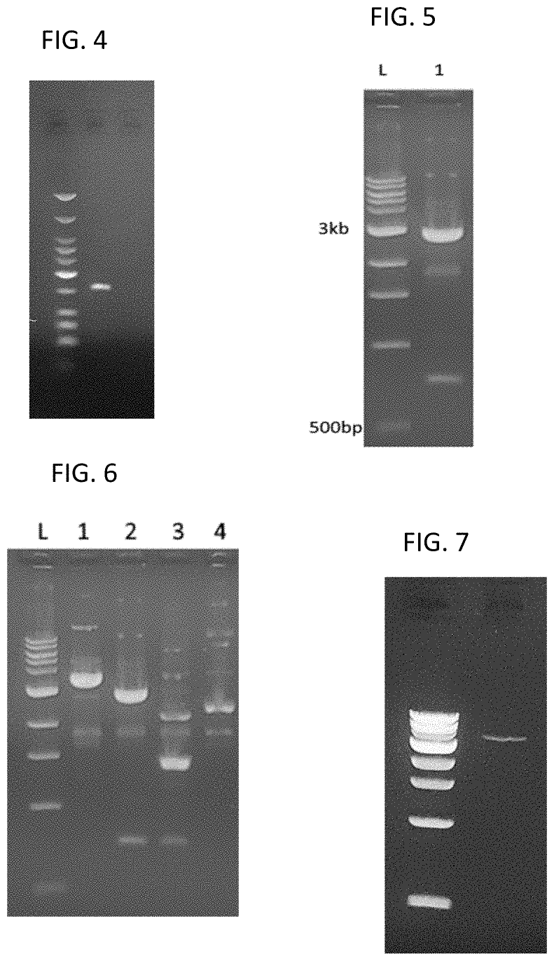

Perform quality analysis of pre-LASSO probe library by running the PCR product on a 2.5% agrose gel and verify the presence of the correct size of the amplicon an optimized PCR-amplified oligo pool yields a strong DNA band/peak at the correct size (FIG. 4). The same analysis can be also performed by suing a Agilent® 2100 Bioanalyzer.

A clean peak at the expected size indicates effective oligo pool amplification. Multiple side peaks indicate non-specific amplification. Repeat PCR with higher annealing temperature to increase specificity, or re-design PCR primers. The presence of a hump after the peak of interest indicates heteroduplexes, a result of over-amplification. Re-try PCR with lower number of cycles

Purify the PCR reactions with AMPure magnetic beads using a high bead-to-DNA ratio (1.8×)

Add 1.8× AMPure magnetic beads (45 μL of beads) to the sample and gently mix. Incubate the sample with the beads at room temperature for 5 min. Condense the beads into a pellet with the magnet for 3-5 min. Remove and discard the supernatant without disturbing the beads, leaving ˜3 μL behind a. Keep the beads pelleted until the elution step; do not disturb the pellet. Pipette 200 μL of 80% (vol/vol) ethanol without disturbing the beads, and keep them pelleted. Leave the ethanol on the beads for 30 sec; then remove and discard the ethanol. Repeat the wash (for a total of two ethanol washes). Remove as much of the ethanol as possible. Air-dry the pellet for ˜1 min.

Add 25 μL of nuclease-free water to the sample and then pipet 15 times to mix. Repeat the mixing to ensure better recovery. Incubate at room temperature for 5 min. Condense beads into a pellet with the magnet for 3-5 min. Collect the supernatant into a new tube Quantify the concentration of the purified PCR product using a Nanodrop. The purified PCR product can be stored at −20° C.

### Example 2

### pLASSO Vector Generation

This example describes methods that can be used to generate a pLASSO vector (e.g., 14 in FIGS. 1A, 2).

In a PCR tube add 2.5 μl, 50 ng of pLox2+ linear plasmid, 1 unit of T4 DNA ligase, nuclease-free water to 25 μl total volume. Add T4 ligase last. Incubate overnight at 16° C. Thaw a vial of 5-alpha chemically competent E. coli cells (New England BioLabs, cat. no. C2989K) on ice and add 50 μL in an ice pre chilled MicroPulser Cuvette 0.1 cm gap. Add 0.5 μl of the overnight ligation reaction and perform electroporation using an electroporator. Subsequently, add 950 μL of 37° C. pre-warmed SOC medium and shake a 200 RPM for 1 h at 37° C. Plate 100 μl of the SOC medium on an ampicillin Agar plate and incubate ON at 37° C. Single colonies from ampicillin agar plate are collected and used to inoculate 5 ml of LB medium with ampicillin in a Corning tube and shake at 200 RPM ON at 37° C. Perform plasmid extraction using the PureLink Quick Plasmid Miniprep Kit as described by the vendor.

The resulting digestion of pLox2+ is incubated with:

### Component

EcoRI restriction enzyme

### Alkaline Phosphatase, Calf Intestinal,

5 μL of CutSmart buffer,
500 ng of pLox2+
Nuclease free water to 25 ul

The reaction is incubated in a thermal cycler at 37° C. for 1 h and then heat inactivated at 80° C. for 10 min. Following amplification, the vector (10 ul of digestion) is analyzed using a 1% agarose gel d run at 100V for 30 min (⅔ of the gel). DNA bands of ˜2.9 kb and ˜750 bp should be present in the gel as shown in FIG. 5. The 2.9 kb DNA fragment of pLox2+ is removed from the gel and purified using a Gel/PCR DNA Fragments Extraction Kit, and the final DNA concentration quantified.

Digest 100 ng of the synthetic dsDNA fragment EcoRI Backbone (a synthetic DNA fragment cloned in pLox2+ to generate pLASSO; see “Backbone” in blue in pLASSO the sequence FIG. 2E) with 1 unit of EcoRI restriction enzyme in 25 ul of 1× CutSmart buffer at 37° C. for one hour and purify by using DNA Purification SPRI Magnetic Beads as described by the vendor, and quantify the final DNA concentration. The 2.9 kb DNA fragment of pLox2+ generated above is ligated with the EcoRI Backbone using the conditions below and incubated at 16° C. overnight.

| | Amount | |
|---|---|---|
| | per 25 μL | Final |
| Component | reaction | concentration |
| 2.9 kp fragment from pLox2 | 40 ng | 1.6 ng/μL |
| EcoR1 Backbone | 10 ng | 0.5 ng/μL |
| 10× T4 DNA Ligase Buffer | 2.5 μL | 1× |
| T4 DNA Ligase | 1 μL | 16 units/μL |
| PCR grade water | Fill to 25 μL | |

The ON ligation reaction (0.5 μL) is used for transformation of 5-alpha chemically competent E. coli cells. Following transformation, cells are gown on an ampicillin resistance selective agar plates. Colonies (up to 5) from the ampicillin selective agar plates are collected and used to inoculate LB medium containing ampicillin and shake at 200 RPM ON at 37° C. From the broth cultures, extract pLASSO performing plasmid extraction using the PureLink Quick Plasmid Miniprep Kit as described by the vendor and quantify the final DNA concentration. The correct assembly of pLASSO is determined by performing digestions of ˜500 ng of pLASSO with SalI, EcoR1, SwaI restriction enzymes, and analyzing the fragments using electrophoresis and a 1% agarose gel (FIG. 6). As shown in FIG. 6, SwaI generates a single fragment of 3205 bp, and EcoRI generates a fragments of 338 bp and 2867 bp, and SalI+Swa1 generate 1627 bp and 1577 bp fragments. The resulting assembled pLASSO vector can be stored at −80° C. (differs from pLASSO 14 of FIGS. 1A, 2A-2C, in that this is the covalently closed form; it does not have the “a” and “b” selectors that are attached by PCR during linearization).

The resulting assembled pLASSO vector is linearized to generate the final pLASSO vector 14 in FIGS. 1A, 2. The following PCR reaction is performed:

| | FINAL | PER 25 μL |
|---|---|---|
| COMPONENT | CONCENTRATION | REACTION |
| 5× KAPA HiFi Fidelity | 1× | 5.0 μL |
| Buffer | | |
| 10 mM each dNTP Mix | 0.3 mM each dNTP | 0.75 μL |
| 10 μM NEB1F Primer | 0.3 μM | 0.75 μL |
| 10 μM NEB1R primer | 0.3 μM | 0.75 μL |
| 0.5 ng of pLASSO | 0.4 ng/μL | 0.5 μL |
| KAPA HiFi HotStart | 0.5 units/reaction | 0.5 μL |
| DNA Polymerase | | |
| (1 unit/μL) | | |
| PCR grade water | — | Fill to 25 μL |

PCR Reaction Conditions

| CYCLING | | | |
|---|---|---|---|
| STEP | TEMPERATURE | | DURATION |
| 1 | Initialization | 4 min at 95° C. | 1× |
| | Denaturation | | |
| 2 | Denaturation | 20 sec at 95° C. | 28 Cycles |
| 3 | Annealing | 20 sec at 60° C. | |
| 4 | Extension | 2 min at 72° C. | |
| 5 | Final Extension | 3 min at 72° C. | 1× |

The correct linearized pLASSO structure is confirmed by analyzing the PCR product on a 0.8% agarose gel. The PCR-linearized pLASSO yields a strong DNA band of ˜3.3 kb (FIG. 7). The PCR-linearized pLASSO can be stored at −20° C.

### Example 3

### Mature Ss DNA LASSO Probe Generation

This example describes methods that can be used to generate a mature ssDNA LASSO probe (e.g., 30 in FIGS. 1A, 2).

### Biological Material

5-alpha chemically competent E. coli cells (New England BioLabs, cat. no. C2989K)
5-alpha Electrocompetent E. coli, high efficiency (New England BioLabs, cat. no. C2987I) Escherichia Coli K12 (strain ATCC 27355)

### Reagents

pre-LASSO library (Twist Bioscience; see SEQ ID NOS: 1-3088 for the design of the pre-LASSO probes)
M13mp18 Single-stranded DNA (New England BioLabs, cat. no. N4040S)
pre-LASSO M13 (the positive control for capture experiments see DNA sequence below SEQ ID NO: 3091)
KAPA HiFi HotStart PCR Kit (Catalog #KK2502)
Omni Klentaq LA (DNA Polymerase Technology cat. 350)
Recombinant Bacteriophage P1 Cre recombinase protein (ABCAM cat. no. ab134845)
Deoxynucleotide (dNTPs) solution Mix (New England BioLabs, cat. no. M0210S)
CutSmart buffer R3101S B7204S)
Cre Recombinase Reaction Buffer (New England BioLabs, cat. no. M0298S NEB, only available with Cre recombinase)
EcoRI HF (New England BioLabs, cat. no. R3101S)
SalI (New England BioLabs, cat. no. R0138S)
BamHI (New England BioLabs, cat. no. R0136S)
SwaI (New England BioLabs, cat. no. R0604)
BspQI (New England BioLabs, cat. no. R0712S)
Nt.BbvCI nicking endonuclease (New England BioLabs, cat. no. R0632S)
T4 DNA Ligase (New England BioLabs, cat. no. M0202S)
Ampligase DNA Ligase (100 units/μl) (Lucigen Corporation cat. no. A0102K)
Ampligase 10× Reaction Buffer (Lucigen Corporation cat. no. A1905B)
Lambda Exonuclease (New England BioLabs, cat. no. M0262S)
Exonuclease V (RecBCD) (New England BioLabs, cat. no. M0345S)
USER enzyme (New England BioLabs, cat. no. M5505S)
Adenosine 5′-Triphosphate (ATP) 10 mM (New England BioLabs, cat. no. P0756S)
NEBNext dsDNA Fragmentase (New England BioLabs, cat. no. M0348S)
Gel/PCR DNA Fragment Extraction Kit (IBI scientific cat. no. 1B47010)
UltraPure Ethidium Bromide, 10 mg/mL (Thermo Fischer Scientific, cat. no. 15585011)
SOC outgrowth medium (New England BioLabs, cat. no. B9020S)
PureLink Quick Plasmid Miniprep Kit (thermos Scientific, cat. no. K210010)
Difco, LB Broth Miller (Luria-Bertani), 500 g (Sigma Aldrich L3522)
pLox2+ (linearized) (it comes together with Cre Recombinase New England BioLabs, cat. no. M0298S)
M13mp18 Single-stranded DNA (New England BioLabs, cat. no. N4040S)

### Equipment

Accuris myGel™ Mini Agarose Gel Electrophoresis Apparatus (Accuris Instruments, cat. no. E1101)
Accuris UV Transilluminator (Accuris Instruments, cat. no. E3000) !CAUTION Always wear UV-light-protective safety glasses/face shield.
Accuris SmartDoc 2.0 Imaging Enclosure (Accuris Instruments, cat. no. E5001-SD)
Accuris SmartDoc 2.0 System with Blue Light Illumination Base, 115V (Accuris Instruments, cat. no. E5001-SDB)
SmartDoc band pass filter, 590 nm, for imaging EtBR on UV transilluminator (Accuris Instruments, cat. no. EE5001-590)
MicroPulser electroporation apparatus (Biorad, cat. no. 165-2100)
Gene Pulser/MicroPulser Cuvette 0.1 cm gap (Biorad, cat. no. 165-2089)
AMPure XP for PCR Purification (Beckman Coulter Life Sciences)

### Reagent Setup

Oligos and primers
Resuspend IDT DNA oligos (SEQ ID NOS: 3104 and 3105 [sap1F and ThiolR above])) and primers to 100 μM in nuclease-free water. Dilute to a 10 μM concentration by adding 10 μL of 100 μM primers to 90 μL of nuclease-free water. DNA oligos and primers can be stored at 10 μM or 100 μM at −20° C. for up to 2 years.

1×TAE Buffer
Mix 100 mL of 10×TAE with 900 mL of water for 1 L of 1×TAE. Store at room temperature (25° C.) until expiration date on packaging.

80% (Vol/Vol) Ethanol Solution
Mix 8 mL of ethyl alcohol (pure, 200 proof) with 2 mL of nuclease-free water to obtain 1 mL of 70% (vol/vol) ethanol right before use.

CRE Recombinase (ABCAM)

Aliquot in PCR tubes in 4 μl aliquots and store at −80° C.

### Gap Filling Mix

Prepare Gap Filling Mix assembling the component with the order shown in table, vortex and store at −20° C. for up to three months

| | | Amount PER |
|---|---|---|
| ORDER | COMPONENT | 1 ml Stock |
| 1 | PCR grade Water | 791 μl |
| 2 | 10X Ampligase | 100 μl |
| | DNA ligase Buffer | |
| 3 | 10 mM dNTPs | 4 μl |
| 4 | Ampligase DNA | 1 μl |
| | Ligase (5 U/ul) | |
| 5 | TaqDNA | 4 μl |
| | Polymerase | |
| 6 | Glycerol | 100 μl |

### Digestion Mix

Prepare Digestion Mix assembling the component with the order shown in table, vortex and store at −20° C. for up to three months

| | | Amount PER |
|---|---|---|
| ORDER | COMPONENT | 1 ml Stock |
| 1 | PCR grade Water | 120 μl |
| 2 | Exonuclease I | 40 μl |
| 3 | Lambda Exonuclease | 40 μl |
| 4 | Exonuclease III | 40 μl |

### Agarose Gel

Mix 0.6 g for 1.2% (wt/vol) agarose with 50 mL of 1×TAE, heat in microwave until agarose completely dissolves, add 1.5 μL of ethidium bromide (10 mg/mL) pour the solution into the casting box with the comb positioned, and cool at room temperature for at least 20 min until the gel solidifies.

### Oligonucleotide List

| Name | Sequence 5′-3′ |
|---|---|
| EcoRI Backbone | TCGAGGAATTCAGAGAAGTCATCAAAGAGTTTAAAGA |
| (SEQ ID NO: 3089) | GTTTATGAGATTTAAGGTCAAGACAACGAGACACGAGT |
| | TCGAGATTGAGGGAGAGAAGGCCCCTCAGCGGCCTTAT |
| | AACTATAACGGTCCTAAGGTAGCGAACGAACAAACCG |
| | CTAAGCTCAAGGTCACAAAAGGTCGACGAGGACCCGG |
| | ATCCCTCCCCTTCTCCTGGTACGGAAGCAAAGCCTATG |
| | TTAAACACTGACTATCTGAAGCTCTCCTTCCCTGAAGG |
| | CTTGAGAGATTCATGAACTTCGAGGAAGGACGGAGAG |
| | TTTATTTATAAGGAACCAACTTCCCCTCCGATGGCCCTG |
| | TCATGAATTCT |
| Pre-LASSO 1 | CAGACGACGGCCAGTGTCGACNAACACTTCTTGCGGCG |
| (SEQ ID NO: 3090) | ATGGTTCCTGGCTCTTCGATCNGGATCCTACGGTCATTC |
| | AGC |
| pre-LASSO M13 | CAGACGACGGCCAGTGTCGACTTGGAGTTTGCTTCCGG |
| (SEQ ID NO: 3091) | TCTGGTTCGAACACTTCTTGCGGCGATGGTTCCTGGCTC |
| | TTCGATCGCCGTTGCTACCCTCGTTCCGATGCGGATCCT |
| | ACGGTCATTCAGC |
| Pre-LASSO GAPDH | CAGACGACGGCCAGTGTCGACGGTGAAGGTCGGAGTCA |
| (SEQ ID NO: 3092) | ACGGATTTGGTCGAACACTTCTTGCGGCGATGGTTCCTG |
| | GCTCTTCGATCGGAAGAGAGAGACCCTCACTGCTGGGG |
| | GGATCCTACGGTCATTCAGC |
| Pre-LASSO F-Actin | CAGACGACGGCCAGTGTCGACATGGAAGAAGAGATCG |
| (SEQ ID NO: 3093) | CCGCGCTGGAACACTTCTTGCGGCGATGGTTCCTGGCTC |
| | TTCGATCCCCCCAGAGCGCAAGTACTCGGTGTGGATCCT |
| | ACGGTCATTCAGC |
| Selector a | CAGACGACGGCCAGTGTC |
| (SEQ ID NO: 3094) | |
| Selector b | GCTGAATGACCGTAGGATCC |
| (SEQ ID NO: 3095) | |
| Selector c | AAATGCGACCCCGAATG |
| (SEQ ID NO: 3096) | |
| Selector d | CTATGGGATGCGATGGGAT |
| (SEQ ID NO: 3097) | |
| Selector e | GTATTGGCAGGGTCTCCG |
| (SEQ ID NO: 3098) | |
| Selector f | GAGGGGTCACACCTCCG |
| (SEQ ID NO: 3099) | |
| Selector g | CGCAAGGAATCTGCCTAACC |
| (SEQ ID NO: 3100) | |
| Selector h | ATTTTCAGATCGCGACCATTG |
| (SEQ ID NO: 3101) | |
| pLASSO | GGATCCTACGGTCATTCAGCCTCCCCTTCTCCTGGTACG |
| linearization a | GAAGCAA |
| (SEQ ID NO: 3102) | |
| pLASSO | GTCGACACTGGCCGTCGTCTGCTTTTGTGACCTTGAGCT |
| linearization b | TAGCGGT |
| (SEQ ID NO: 3103) | |
| SapIF | GGTTCCTGGCTCTTCGATC |
| (SEQ ID NO: 3104) | |
| ThiolR | A*T*C*GCCGCAAGAAGTGTU (indicates phosphorothioate |
| (SEQ ID NO: 3105) | bonds) |
| PostCaptR | CTTCCGTACCAGGAGAAGGG |
| (SEQ ID NO: 3106) | |
| PostCaptF | ACCGCTAAGCTCAAGGTCACA |
| (SEQ ID NO: 3107) | |
| Neb1F | AGCCTCCCCTTCTCCTGGGATCCTACGGTCATTCGTACG |
| (SEQ ID NO: 3108) | GAAGCAA |
| Neb1R | TTTTGTGACCTTGAGCTTAGCGGTGTCGACACTGGCCG |
| (SEQ ID NO: 3109) | TCGTCTGC |

Software: pre-LASSO calculator software

### Procedure

Cloning in pLASSO
1. Thaw on ice with the pre LASSO library pre-amplified as described in Example 1 and pLASSO obtained as described in Example 2

In parallel with the assembly of the LASSO library(s) perform, in a separate tube, the assembly LASSO M13 starting from pre-LASSOM13 (SEQ ID NOS3104 and 3105) and pLASSO linearized with NEB1F and NEB1R primers (SEQ ID NOS: 3108 and 3109). LASSO M13 will be used as positive control for subsequent capture experiments. Since pre lasso pre-LASSOM13 is purchased as a dsDNA oligo (Gblock, IDT) it does not need to be pre-amplified, thus start the assembly directly from the cloning at step 25 below
2. For each pre-LASSO library set up a PCR with the following NebBuilder assembly reaction. Include a separate tube for pre-LASSO M13

### Neb Builder Reaction Components

| | | PER 20 μL |
|---|---|---|
| COMPONENT | Amount | REACTION |
| Linearized pLASSO | ~50 ng | 2.5 ng/μL |
| Pre-LASSO library | ~16 ng | 0.8 ng/μL |
| (pre-LASSO M13) | | |
| NEBuilderHiFi DNA Assembly | 10 μL | 1X |
| Master Mix 2X | | |
| PCR grade water | Fill to 20 μL | |

3. Incubate in a PCR thermal cycler at 50° C. for 15 minutes. Following incubation, store samples on ice or at −20° C. for subsequent transformation

E. coli Transformation
4. Prepare LB agar plates with ampicillin (optimally by dispensing 40 ml of LAB agar 100 μg/ml ampicillin). Once the once the agar is solidified incubate at 37° C.
5. Thaw NEB 5-alpha electro competent cells on Ice. Transfer 50 μL of electro competent cells to a pre-chilled electroporation cuvette with 1 mm gap, Add 1 μL of the assembly product above to electro competent cells. Mix gently by pipetting up and down. Once DNA is added to the cells, electroporation immediately. Add 950 μL of room-temperature SOC media to the cuvette immediately after electroporation. Place the tube at 37° C. for 60 minutes. Shake vigorously (250 rpm) or rotate. Warm selection plates to 37° C. Include a pUC19 NEB positive control for electroporation (provided with electrocompetent cells)
6. Plate 900 μL of the SOC medium containing transformed E. coli cells in two pre warmed petri dishes (2×-450 μL) and incubate overnight at 37° C. Use the remaining ˜100 μL volume to make 1/10 and 1/100 serial dilutions in fresh SOC medium and plate the 1/10 and 1/100 in smaller petri dishes and Incubate overnight at 37° C.
7. The day after estimate the number of colonies in the petri dishes by counting the E. coli colonies in the dilution plates.

To ensure a uniform representation of all probes in the final LASSO library, the number of the E. coli colonies in selection agar plates should be 10 times the number of pre-LASSO probes in the library (e.g., a 4000 different pre-LASSO probe library needs 40,000 colonies). If the total number of colonies is lower than 10 times the number of pre-LASSO probes, go back to step 5, perform multiple electroporations to reach the required number of colonies and plate in a larger number of petri dishes.

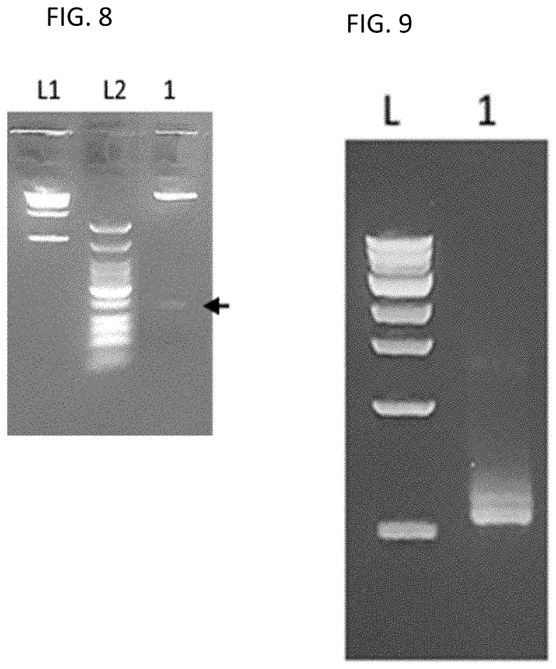

If the number of colonies in the dilution plate is too low whereas the pUC19 control plate have high number of colonies double check that the pLASSO was linearized by using the correct adapters for the pre-LASSO library of choice. Verify identity, purity and concentration of both linearized pLASSO and pre-LASSO library.
8. Harvest E. coli colonies from agar plates by spreading ˜10 ml or larger volume of sterile water on selection agar plates, scrape colonies by using a glass or a plastic spreader. Collect the E. coli solution and dispense the same library in a single 50 ml Corning tube.
9. Pellet the E. coli cells by centrifugation and resuspend the cell in Resuspension Buffer R3 (PureLink quick Plasmid Miniprep Kit) by using 250 μl of R3 Buffer every 5 ml of the E. coli solution. Dispense the resuspended cells in 300 ul aliquots in 1.5 ml Eppendorf tubes than follow the lysis protocol as described by the Invitrogen PureLink quick Plasmid Miniprep Kit.
10. Quantify the concentration of the eluted library. Can store at −20° C.
11. Verify successful cloning of the pre-LASSO pool into pLASSO by setting up a double digestion (see table below) in 25 μl of 1× cut Smart Buffer using 500 ng of the recovered pLASSO library, 1 μl of SalI and 1 μl BamHI. Digest for 1 h at 37° C. Perform gel electrophoresis by loading 4 μL of the digestion in a 2% agarose gel. If the cloning of the pre-LASSO library was successful, a DNA band having the size of the pre-LASSO library (˜160 bp) should be present (FIG. 8).
   Components for the pLASSO Digestion

| | | | FINAL |
|---|---|---|---|
| COMPONENT | | | CONCENTRATION |
| pLASSO cloned library | 500 | ng | 2.5 ng/μL |
| CutSmart Buffer | 2.5 | μL | 1X |
| Sail restriction enzyme | 20 | units | 0.8 units/μl |
| BamHI restriction enzyme | 20 | units | 0.8 units/μl |
| Nuclease free water | | | Fill to 25 μL |

### Nicking

12. Perform nicking endonuclease digestion of the pLASSO library as follows

| | | FINAL |
|---|---|---|
| COMPONENT | AMOUNT | CONCENTRATION |
| pLASSO library | 2 μg | 4 ng/μL |
| CutSmart Buffer | 5 μL | 1X |
| Nt.BbvCI (10 units/μL) | 1 μL | 0.4 U/μl |
| Nuclease free water | Fill to 50 μl | |

Gently mix the reaction and incubate at 37° C. for 1 h. Use the concentration measured at 10 for next step. Can store at −20° C.

Cre Recombination and Purification of DNA Minicircles
13. Perform the Cre recombination of the nicked pLASSO library in 12, as shown in the table below.

| | | FINAL |
|---|---|---|
| COMPONENT | AMOUNT | CONCENTRATION |
| Niked pLASSO library | 25 ng | 5 ng/μL |
| Cre Recombinase Buffer | 5 μl | 1X |
| (NEB) | | |
| Cre Recombinase | 1 μl | 0.01 μg/μl |
| (ABCAM) | | |
| (0.5 mg/ml) | | |
| Nuclease free water | Fill to 50 μl | |

14. Gently mix the reaction and incubate at 37° C. for 30 min.
15. Heat-inactivate at 70° C. for 10 min
16. Add 1 μl of SwaI directly to the 50 μl Cre-Recombinase reactions in
17. Gently mix the reaction by pipetting and incubate at 25° C. for 1 h
18. Heat-inactivate at 70° C. for 10 min
19. Cool the reaction on ice
20. Add 2 μl ATP 10 mM and 1 μl di Exonuclease V
21. Gently mix the reaction and incubate at 37° C. for 30 min
22. Heat-inactivate at 70° C. for 30 min. Can Store at −20° C.

Inverted PCR
23. Use 10 μl of the solution in 22 from as template for the following PCR reaction

| | FINAL | PER 25 μL |
|---|---|---|
| COMPONENT | CONCENTRATION | REACTION |
| DNA solution | — | 10 μL |
| 10 mM each dNTP Mix | 0.3 mM each dNTP | 1 μL |
| 10 μM TiolForward | 0.3 μM | 1.5 μL |
| Primer | | |
| 10 μM SapI Reverse | 0.3 μM | 1.5 μL |
| primer | | |
| KAPA HiFi HotStart | 0.04 units/μL | 1 μL |
| DNA Polymerase (1 | | |
| unit/μL) | | |
| 5x KAPA HiFi Fidelity | 1x | 10 μL |
| Buffer | | |
| PCR grade water | | 25 μL |

PCR Reaction Conditions

| CYCLING | | | |
|---|---|---|---|
| STEP | TEMPERATURE | | DURATION |
| 1 | Initialization | 3 min at 95° C. | 1x |
| | Denaturation | | |
| 2 | Denaturation | 20 sec at 98° C. | 25 Cycles |
| 3 | Annealing | 15 sec at 60° C. | |
| 4 | Extension | 20 sec at 72° C. | |
| 5 | Final | 1 min at 72° C. | 1x |
| | Extension | | |

24. Add 4 μL of the PCR product in a new PCR tube and add 1.5 μL of 6× loading dye and load on a 1.2% (wt/vol) EtBr agarose gel (in 1×TBE) at 100V for 30 min
25. Illuminate the DNA in the gel with a UV transilluminator. The expected PCR product is a strong DNA ˜550 bp band expected for the mature LASSO probes (FIG. 9). The same analysis can be also performed by using an Agilent® 2100 Bioanalyzer.
26. Place AMPure magnetic beads and at room temperature for 30 min and vortex before use.
27. Add 1.8× AMPure magnetic beads (83 μL of beads for the remaining 46 μL of inverted PCR reaction) to the sample and gently mix.
28. Incubate the sample with the beads at room temperature for 5 min.
29. Condense the beads into a pellet with the magnet for 3-5 min.
30. Remove and discard the supernatant without disturbing the beads, leaving ˜3 μL behind. Keep the beads pelleted until the elution step; do not disturb the pellet.
31. Pipette 200 μL of 80% (vol/vol) ethanol without disturbing the beads, and keep them pelleted.
32. Leave the ethanol on the beads for 30 sec; then remove and discard the ethanol.
33. Repeat the wash (for a total of two ethanol washes).
34. Remove as much of the ethanol as possible.
35. Air-dry the pellet for ˜1 min.
36. Add 25 μL of nuclease-free water to the sample and then pipet 15 times to mix. Repeat the mixing to ensure better recovery.
37. Incubate at room temperature for 5 min.
38. Condense beads into a pellet with the magnet for 3-5 min.
39. Collect the supernatant into a new tube
40. Quantify the concentration of the purified PCR product

Maturation
41. Add to the PCR tube in 61 2.5 uL of CutSmart Buffer, 2 uL of BspQI restriction enzyme
42. Gently mix and incubate at 50° C. for 1 h
43. Heat-inactivate for 20 min at 80° C.
44. Add 1 uL of Lambda Exonuclease
45. Gently mix and incubate for 30 min at 37° C.
46. Heat-inactivate for 10 min at 80° C.
47. Add 2 μL of USER enzyme
48. Gently mix and incubate at 37° C. for 30 min
49. Store the mature LASSO probe library at −20° C.
50. Store −20° C. the mature LASSO M13 probe that will be used as positive control for capture experiments

Capture
200-500 ng of bacterial total genomic DNA can be used for a single capture experiment. For eukaryotic genomes, at least to 1-2 μg total genomic DNA or cDNA can be used for a single capture. Consequently, the DNA template needs to be of the appropriate concentration in order to fit the 15 μL capture volume. For bacterial or small genomes ˜50 ng/μL concentration can be sufficient. For eukaryotic DNA or cDNA at least ˜250 ng/μL of template DNA can be used.
To increase capture efficiency and signal to noise ratio, genomic DNA can be fragmented. Exemplary fragment size distribution ranges from 1 kb to 10 kb. Fragmentation can be performed by using a sonication device such as a Covaris or NEBNext dsDNA Fragmentase.
1. In the PCR thermal cycler set up the following:

| CYCLING | | | DURATION |
|---|---|---|---|
| STEP | TEMPERATURE | | (CYCLE) |
| 1 | Denaturation 1 | 5 min at 98° C. | 1x |
| 2 | Hybridization | 60 min* at 65° C. | 1x |
| 3 | Add Gap filling | 5 min at 65 65°C | 1x |
| | Mix | | |
| 4 | Capture | 30 min at 65° C. | 1x |
| 5 | Denaturation 2 | 5 min at 98° C. | 1x |
| 6 | Add Digestion | 5 min at 37° C. | 1x |
| | MIX | | |
| 7 | Digestion | 30 min at 37° C. | 1x |
| 8 | Inactivation | 20 min at 80° C. | 1x |
| 9 | End | ∞ 4° C. | 1x |

* In some examples, 60 min of hybridization is optimal for bacterial genomes

For eukaryotic or human DNA capture, overnight hybridization can be performed.
2. Obtain LASSO M13 positive control, M13mp18 Single-stranded DNA, desired LASSO probe library(es) and DNA template.
3. Dilute the LASSO M13 positive control for capture 1/10 and 1/100 (vol/vol) in PCR grade water
4. Prepare positive and negative control capture below Positive control Capture Reaction Components

| | | FINAL |
|---|---|---|
| COMPONENT | AMOUNT | CONCENTRATION |
| LASSO probe M13 (1/100) | 1 μL | — |
| M13mp18 Single-stranded DNA | 0.5 μl | 0.03 ng/μL |
| 10X Ampligase DNA Ligase | 1.5 μl | 1X |
| Buffer | | |
| PCR grade water | Fill to 15 μl | — |

Negative control Capture Reaction Components

| | | FINAL |
|---|---|---|
| COMPONENT | AMOUNT | CONCENTRATION |
| LASSO probe M13 (1/100) | 1 μL | — |
| 10X Ampligase DNA Ligase | 1.5 μl | 1X |
| Buffer | | |
| PCR grade water | Fill to 15 μl | — |

5. Set up the capture reaction(s) as follows in a PCR tube rack at room temperature
Capture n1 . . . n2

Library Capture Reaction Components

| | | FINAL |
|---|---|---|
| COMPONENT | AMOUNT | CONCENTRATION |
| Mature LASSO probe library | 10 ng | 0.7 ng/μL |
| Fragmented DNA template* | up to 2 μg** | 133 ng/μL |
| 10X Ampligase DNA Ligase | 1.5 μl | 1X |
| Buffer | | |
| PCR grade water | Fill to 15 μl | — |

6. In a thermal cycler, the capture reactions is subjected to DNA denaturation. After denaturation the LASSO probe library hybridizes with the DNA template. After hybridization, 5 μl of the “Gap filling Mix” are added to the capture reaction. DNA target Capture is performed for 30 min at 65° C. After the capture (30 min), the temperature is lowered to 37° C. and immediately, 3 μl of Digestion Mix” are added in solution. Digestion is performed for 1 h at 37° C. followed by exonuclease inactivation at 80 for 20 min.

Post Capture PCR
6. Prepare and run the following PCR reaction

| | FINAL CON- | PER 50 μL | |
|---|---|---|---|
| COMPONENT | CENTRATION | REACTION | |
| Capture Reaction | — | 10 μL | |
| 10 mM each dNTP Mix | 0.3 mM each | 1 μL | |
| | dNTP | | |
| 10 μM PostCaptF primer | 0.3 μM | 1.5 μL | |
| 10 μM PostCapR primer | 0.3 μM | 1.5 μL | |
| Omni Klentaq LA | units/μL | 0.5 μL | |
| 10 x Klentaq DNA | 1x | 5 μL | |
| Polymerase Buffer | | | |
| PCR grade water | | 30.5 μL | |
| CYCLING | FINAL | | PER 50 μL |
| STEP | TEMPERATURE | | REACTION |
| 1 | Initialization | 3 min at 95° C. | 1x |
| | Denaturation | | |
| 2 | Denaturation | 20 sec at 98° C. | 25 Cycles |
| 3 | Annealing | 15 sec at 60° C. | |
| 4 | Extension | 20 sec at 72° C. | |
| 5 | Final | 1 min at 72° C. | 1x |
| | Extension | | |

### Example 4

### Mature Ss DNA LASSO Probe Generation

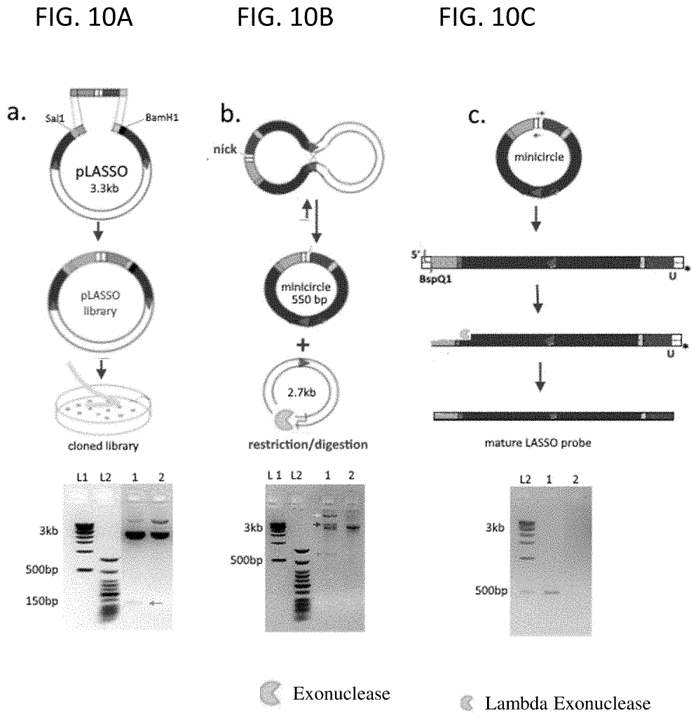

Schematics of an exemplary embodiment of the disclosed assembly methodology is shown in FIG. 10A-10C. A single pre-LASSO probe or a pre-LASSO library in shuttled in the linearized pLASSO vector via a Gibson Assembly or by using NEBuilder DNA Assembly Master Mix (NEB) and used for transformation in E. coli. The cloned library is harvested by scraping a sufficient number of colonies from plates. Plasmids are purified by using a plasmid miniprep. The presence of the pre-LASSO probes in the plasmids was verified by digesting with restriction enzymes that cut adjacently to the Gibson assembly insertion sites (Sal1, BamH1 sites). Gel electrophoresis results illustrate successful cloning of the pre-LASSO library in pLASSO.

As shown in FIG. 10B, the native supercoiled plasmids obtained by colony miniprep, are converted in the relaxed form by nicking with endonuclease Nt.BspQ1 that uses a recognition site located in the primer annealing site of the inserted pre-LASSO probe. Cre recombination of the LoxP sites produces a DNA minicircle containing the pre-LASSO and a circular 2.7 kb DNA circle, the remaining part of pLASSO. After recombination, the 2.7 kb DNA circle, together with the unreacted plasmids and bigger DNA circles generated by inter-plasmid recombination (not shown) are eliminated by restriction followed by exonuclease digestion.

Gel electrophoresis results illustrate successful formation of the expected DNA minicircles (orange arrow) together with the 2.7 kb circular DNA remaining parts of pLASSO (green arrow), the unreacted plasmid (blue arrow). The approximately 6 kb band (yellow arrow) correspond to the recombination of two different plasmids (inter-plasmid recombination). When using the natural un-nicked pLASSO library form for Cre recombination the DNA band correspondent to DNA minicircle was absent (Lane 2) indicating that nicking mediated pLASSO plasmid relaxation helped to ensure efficient Cre-recombination. Relaxation of pLASSO plasmid induced by cutting one of the two DNA strands may allow the two recombination sites to be in closer proximity thus resulting in a more efficient formation of the Cre-recombinase synapse tetramer in which four distinct active sites are present.

### Example 5

### LASSO Probe Performance and Sensitivity Test

To assess the ability of LASSO probes in capturing a DNA target of various length, the disclosed methods were used to assemble two 550 bp mature LASSO probes containing arms designed to capture of 1 kb and 4 kb DNA target regions within ˜7.5 kb the genome of the M13mp18 phage. The sequence of the two LASSO probes were verified using Sanger sequencing. Capture experiments were performed by following the previously developed capture procedure as described by Tosi et al. (Nat Biomed Eng. 2017; 1:0092, 2017).

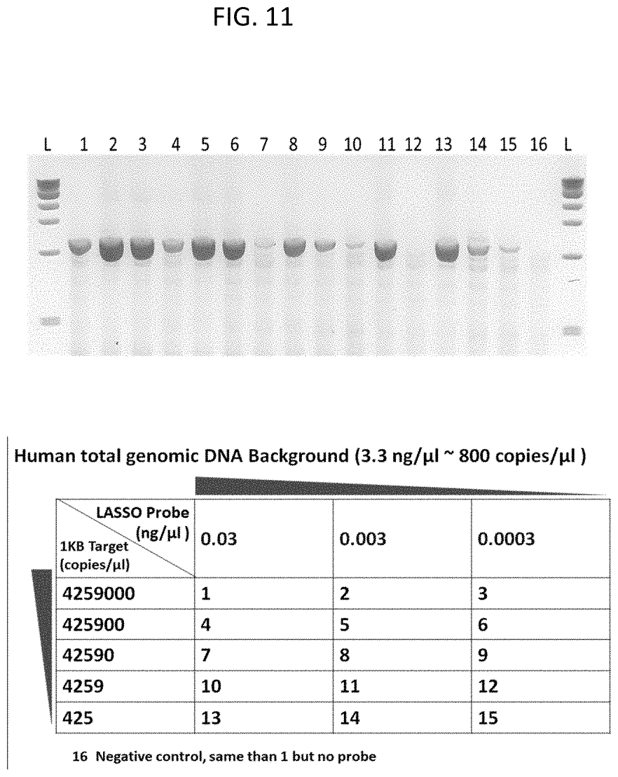

The post capture PCR amplicons of the expected 1 kb and 4 kb sizes were present (FIG. 11) indicating successful capture. To test the feasibility of performing a massively multiplexed capture that include thousands of LASSO probes (individually at low concentration) in the human genome, a series of capture reactions were performed in a constant human genomic DNA background where consecutive tenfold dilutions of a single 1 kb target sequence were spiked. The capture of the target sequence was also performed by testing progressive tenfold dilutions of the LASSO probe according to the table in FIG. 11. As shown in FIG. 11 the expected capture band was observed even when testing the lowest dilution of the probes with the lowest dilution of the target sequence. In this latter condition, in the 15 μl capture volume, there were only 4*10-3 ng of the single LASSO probe and the molarity of the targeted 1 kb sequence was half of the molarity of the human genomic DNA background (500 ng correspondent to ˜400 copies/μl). “off target” products were not observed when the target sequence was absent from the reaction, thus highlighting the specificity of the reaction. These results demonstrate that a very large LASSO library (composed of hundreds of thousands of probes) can fit in nanograms of total DNA library and the capture reaction is sensitive enough for massive parallel target capture in the human genome.

### Example 6

### Disclosed Versus Previous LASSO Assembly Methods

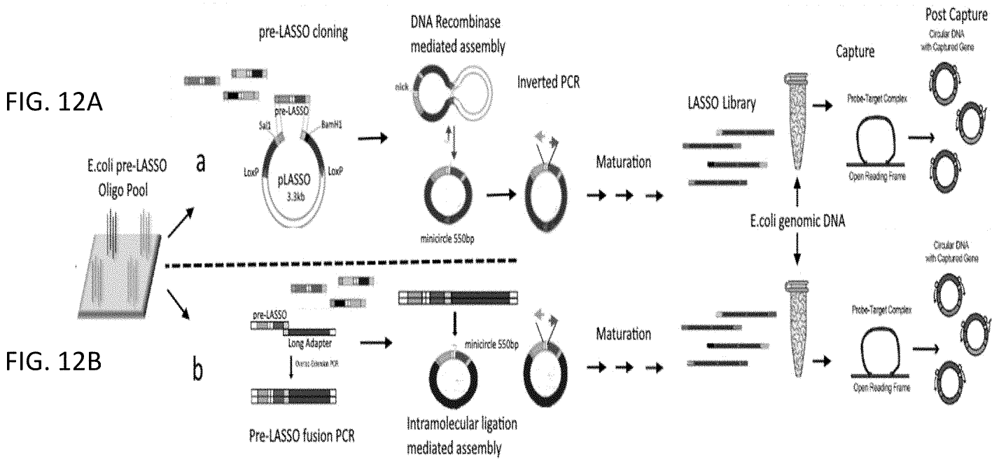

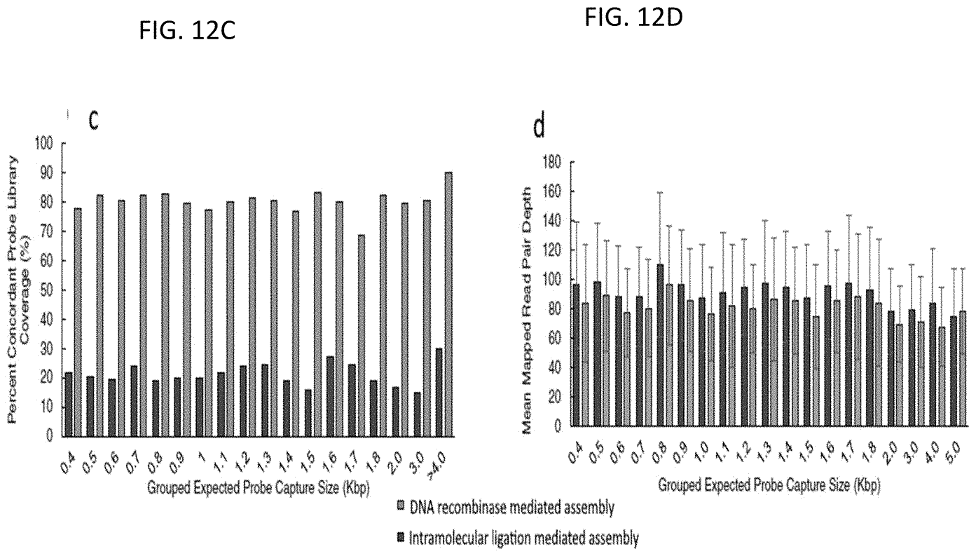

The performance of LASSO probes assembled using the disclosed DNA recombinase mediated methodology (FIG. 1A) to LASSO probes assembled using the previous Intramolecular ligation assembly methodology (FIG. 1B) in capturing a library of kilobase-sized ORFs from E. coli genomic DNA was compared. A schematic of the workflow of the LASSO assembly and capture experiment is presented in FIG. 12A-12B.

The ssDNA pre-LASSO probes were obtained from Twist Bioscience as a single oligo pool composed by 3078 pre-LASSO probes. The pre-LASSO probe had the exact same arm design of the pre-LASSO probe previously developed (Tosi et al. 2017). Of the 3,664 pre-LASSO probes those corresponding to ORF targets smaller than 400 bp were removed as a precaution to avoid potentially skewing the capture library during its subsequent PCR amplification and an additional 160 probes were also removed that targeted different capture targets lengths as negative control. Adjusting the thresholds for target length, melting temperature or the length of the ligation/extension arms determines the number of acceptable probes. Approximately 22.5% of the E. coli K12 ORFeome (900 ORFs) was thus left untargeted and used as an internal, negative control for our experiments. The E. coli LASSO probe library was assembled according with the protocol described herein.

The pre-LASSO ssDNA oligo pool was converted to dsDNA format by performing 8 PCR cycles with selector primers and cloned inserted in pLASSO by using NEBuilder HiFi DNA Assembly and transformed in electro-competent E. coli cells. Approximately ˜40,000 E. coli colonies were scraped from antibiotic agar plates representing 10× coverage of the LASSO probes contained in the E. coli library. The pLASSO library was extracted by plasmid miniprep and subjected to recombination with the Cre-recombinase enzyme. The circular LASSO precursors (DNA minicircles) were linearized by inverted PCR and underwent maturation as described above.

At the end of the inverted PCR stage, after DNA column purification, a 5p aliquot of the PCR amplicon was collected for subsequent Illumina NextSeq 150 bp paired ends sequencing in order to assess quality and uniformity of the LASSO library. At the inverted PCR stage, ligation and extension arms are already coupled with the conserved DNA Backbone in the final configuration.

The NGS results were compared to the results previously obtained by Syukri (2019) when assessing the quality of the E. coli LASSO library obtained by using two different dilution volumes for probe circularization.

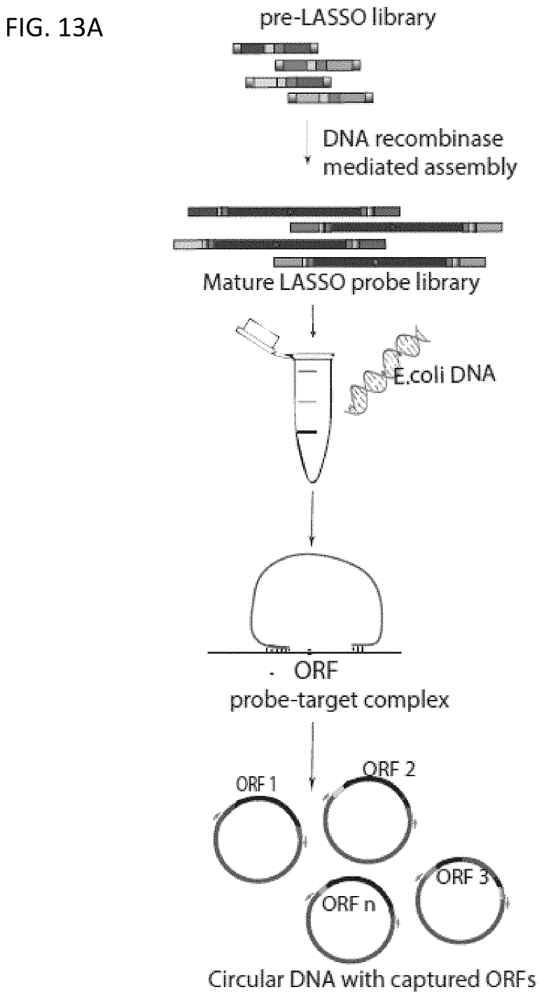

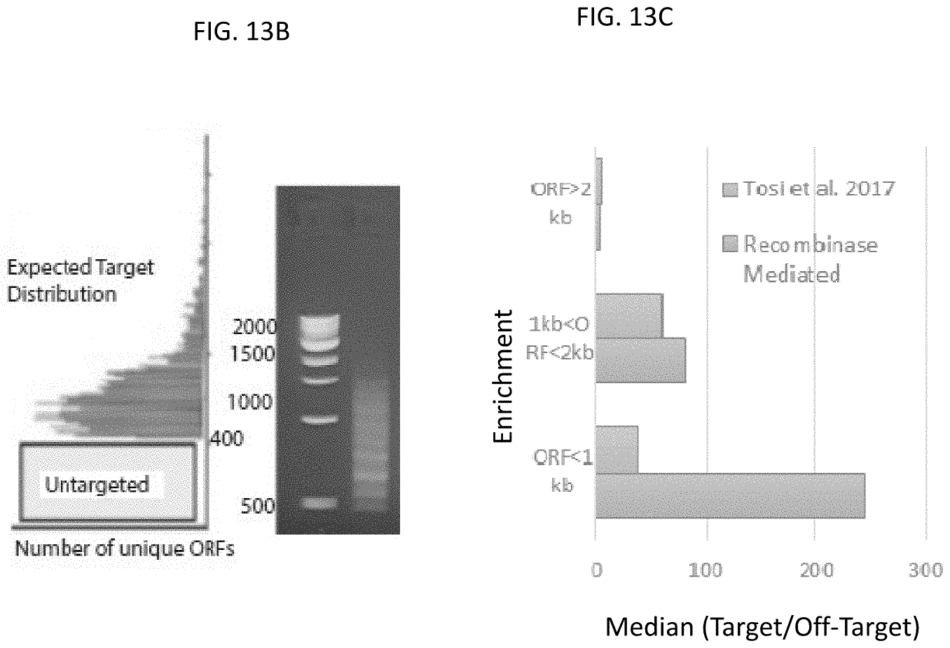

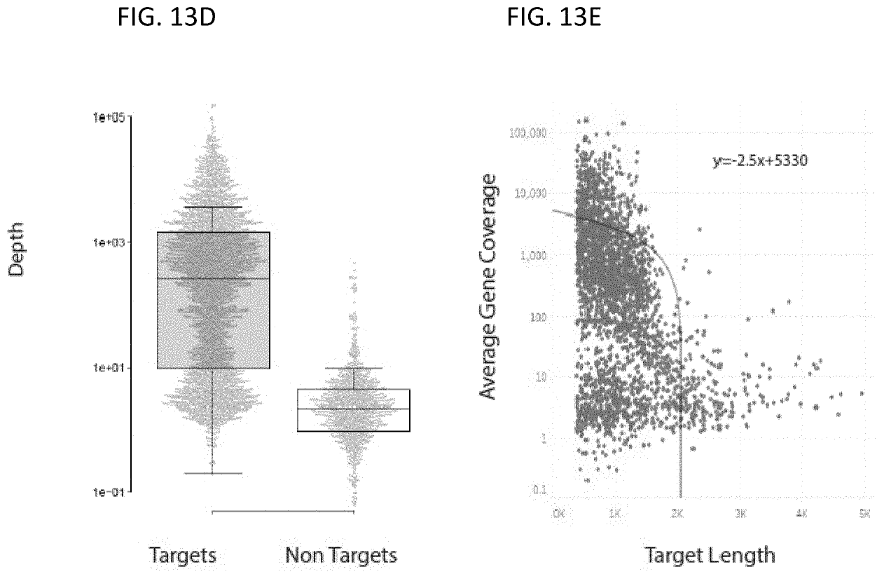

The DNA Recombinase Mediated Assembly resulted in a superior quality of the LASSO library with an average percentage of “arm concordancy” (defined as the percentage of correctly paired probe arms versus total read sequences per probe type) of 40% as shown in FIG. 13A.

The uniformity of the library was assessed by counting the number of the different types of concordant LASSO probes present in the library. As shown in FIG. 13B, the majority of the probes were present within tenfold the normalized abundance of the median indicating a relatively uniform representation of single LASSO probes in the LASSO library.

We next evaluated the ability of the new LASSO probes to capture a library of kilobase-sized ORFs from E. coli genomic DNA using the same capture parameters described by Tosi et al. (2017) including the same amount of LASSO library, and E. coli DNA template. Briefly, LASSO probes were hybridized with total genomic DNA of E. coli K12, targeting the 3078 ORFs in a single reaction volume. Circles containing ORFs were PCR amplified using primers that hybridize to the conserved adapter region on each LASSO probe. Post capture PCR of circles obtained from the capture of 3078 ORFs of E. coli K12 was run in an 1.2% agarose gel and is shown in FIG. 13C. and their apparent size distribution corresponded well with that of the targeted ORFs. The rest of the post capture PCR amplicon was enzymatically shared and sequenced on an Illumina NextSeq instrument to obtain 150 nucleotide paired end reads.

For reads mapping to the E. coli genome, target enrichment factors were calculated, which were defined as the reads per kilobase of genetic element per million reads (RPKM), which were mapped to the targeted ORFs versus non-targeted ORFs. Furthermore, RPKM targeted/non-targeted ratios were analyzed for different length genetic elements by binning FIG. 13D). In this experiment, LASSO targeted ORFs were enriched in all bins (up to ˜250× for ORFs<1 kb) representing 8 times improvement in comparison to enrichment previously measured by Tosi et al. (2017).

FIG. 13E illustrates the distribution of read counts per kilobase for each targeted ORF, each untargeted ORF. The targeted ORFs were significantly enriched compared with the non-targeted ORFs and intergenic regions (by Welch two-sample t-test). The mean and the median RPKM of the targets was 2476 and 264 for the targets respectively while the mean ant the median RPKM of the Non Targets was 31. and 1.26 respectively. Fold-enrichment of targets was calculated to be between 80- and 200-fold (by the median or mean of the target RPKM, respectively, over the mean non-target RPKM). A negative correlation was observed between the normalized abundance of each target ORF and its length; ORF representation was observed to decline by 60% with each doubling of length (FIG. 13F). This bias that was previously reported (Tosi et al. 2017) may reflect target length-dependent capture efficiency, post-capture PCR bias or a combination of the two effects.

### Example 7

### Materials and Methods

This example provides the materials and methods for the results describe below in Examples 8-12.

Design of Single Pre-LASSO Probes that Target M13mp18 Bacteriophage Sequences

Pre-LASSO probe pools are short DNA oligo pools (˜160-180 bp) designed in silico and ordered from Twist Bioscience, then used for the assembly of LASSO probes. Pre-LASSO probes have five different regions: primer-annealing site, ligation arm, conserved region, extension arm, primer-annealing site. The ligation and extension arms of the pre-LASSO probes are designed to have the same 5′-3′ orientation of the sequence of the target DNA.

As a positive control, the same pre-LASSO probe targeting a 1 Kb target capture on the ssDNA of M13mp18 as the one listed by Chkaiban et al. (Curr Protoc, (11):e278, 2021) was used. It had the Tm of the extension arms ˜65° C. and the Tm of the ligation arms ˜70° C. Pre-LASSOs targeting 3 Kb sequences within the M13mp18 genome were manually designed with Tm of the extension arms ˜65° C. and 3 different Tm of the ligation arms 65° C., 70° C. and 75° C.

pre-LASSO probes targeting 4 and 5 kb sequences within the single strand M13mp18 DNA were manually designed with Tm of the extension arms ˜65° C. and the Tm of the ligation arms ˜70° C. The sequences for the above cited pre-LASSO targeting on the M13mp18 genome are listed below. The ligation and extension arms are underlined.

| Name | Sequence 5′-3′ |
|---|---|
| pre-LASSO | SEQ ID NO: 3091 |
| 1kbM13 | |
| pre-LASSO | CAGACGACGGCCAGTGTCGACTTGGAGTTTGCTTCCGGTCTGGTTCGAACACT |
| 3kbM13-65° C. | TCTTGCGGCGATGGTTCCTGGCTCTTCGATCGCTATTGGGCGCGGTAATGATT |
| | GGATCCTACGGTCATTCAGC (SEQ ID NO: 3117) |
| pre-LASSO | CAGACGACGGCCAGTGTCGACCCTGACCTGTTGGAGTTTGCTTCCGGTCTGG |
| 3kbM13-70° C. | TTCGCTTTGAAGCAACACTTCTTGCGGCGATGGTTCCTGGCTCTTCGATCGCT |
| | ATTGGGCGCGGTAATGATT GGATCCTACGGTCATTCAGC (SEQ ID NO: 3118) |
| pre-LASSO | CAGACGACGGCCAGTGTCGACGGTACTCTCTAATCCTGACCTGTTGGAGTTT |
| 3kbM13-75° C. | GCTTCCGGTCTGGTTCGCTTTAAGCTCGAATTAAAACGCAACACTTCTTGCG |
| | GCGATGGTTCCTGGCTCTTCGATCGCTATTGGGCGCGGTAATGATTGGATCC |
| | TACGGTCATTCAGC (SEQ ID NO: 3119) |
| pre-LASSO | CAGACGACGGCCAGTGTCGACTTGGAGTTTGCTTCCGGTCTGGTTCGAACAC |
| 4kbM13 | TTCTTGCGGCGATGGTTCCTGGCTCTTCGATCGGCGAATCCGTTATTGTTTCT |
| | CCCGATGTAGGATCCTACGGTCATTCAGC (SEQ ID NO: 3120) |
| pre- | CAGACGACGGCCAGTGTCGACCCTGACCTGTTGGAGTTTGCTTCCGGTCTGG |
| LASSO5kbM13 | TTCGCTTTGAAGCAACACTTCTTGCGGCGATGGTTCCTGGCTCTTCGATCCCAT |
| | TCAAAAATATTGTCTGTGCCACGTATTCTTACGCGGATCCTACGGTCATTCAGC |

Design of Different Melting Arms the Pre-LASSO Probes Pools for an E. coli Model

The effect of varying the melting temperature of LASSO probes arms on capture efficiency and specificity was achieved by designing probes that targets E. coli ORF's ranging from 999 bp-2000 bp. Specifically, five different pools were generated: a pool that had a 5° C. lower ligation arm (65-70° C.) melting temperature with respect to the extension arm (70-75° C.) (L65E70), a pool that had a 10° C. lower ligation arm (60-65° C.) melting temperature with respect to the extension arm (70-75° C.) (L60E70), a pool that had a 5° C. lower extension arm (65-70° C.) melting temperature with respect to the ligation arm (70-75° C.) (L70E65), a pool that had a 10° C. lower extension arm (60-65° C.) melting temperature with respect to the ligation arm (70-75° C.) (L70E60), and a pool that had extension and ligation arm (65-70° C.) melting temperature in the same range (L65E65). The bio-python based algorithm listed in Chkaiban et al. (Curr Protoc, (11):e278, 2021) was modified by prolonging the arms until the desired melting temperatures were reached and selected probes that would capture E. coli ORF targets ranging from 999 bp to 2000 bp. The bio-python algorithm was performed on the E. coli str. k-12 substr. mg1655 reference ORFeome found in NCBI (RefSeq: NC_000913.3). The new biopython algorithms as well as the resulting pre-LASSO list of probes can be found in the supplementary files.

### Assembly of the LASSO Probes

The assembly of the LASSO probes was performed using a 350 bp backbone according to the protocol described by Chkaiban et al. (Curr Protoc, (11):e278, 2021) for all single LASSOs and LASSO pools. In addition to the assembly with 350 bp backbone, to assess the effect of backbone length on capture efficiency, LASSO probes that target 3 Kb sequences in the M13mp18 bacteriophage were assembled using a longer 700 bp backbone linker. The 700 bp backbone linker was substituted to the 350 bp backbone in the support protocol 1 in the pLASSO plasmid generation listed in Chkaiban et al. (Curr Protoc, (11):e278, 2021) ahead of the LASSO probe assembly protocol. The backbone linker oligonucleotides are listed below.

| Name | Sequence 5′-3′ |
|---|---|
| EcoR1 | TCGAGGAATTCAGAGAAGTCATCAAAGAGTTTAAAGA |
| Backbone- | GTTTATGAGATTTAAGGTCAAGACAACGAGACACGAG |
| 350 bp | TTCGAGATTGAGGGAGAGAAGGCCCCTCAGCGGCCTT |
| (SEQ ID | ATAACTATAACGGTCCTAAGGTAGCGAACGAACAAAC |
| NO: 3122) | CGCTAAGCTCAAGGTCACAAAAGGTCGACGAGGACCC |
| | GGATCCCTCCCCTTCTCCTGGTACGGAAGCAAAGCCT |
| | ATGTTAAACACTGACTATCTGAAGCTCTCCTTCCCTG |
| | AAGGCTTGAGAGATTCATGAACTTCGAGGAAGGACGG |
| | AGAGTTTATTTATAAGGAACCAACTTCCCCTCCGATG |
| | GCCCTGTCATGAATTCT |
| EcoR1 | TCGAGGAATTCAGAGAAGTCATCAAAGAGTTTAGTGA |
| Backbone- | GGCTCGTCCATCTGACGGCTGCTCATTGGTGTGGCTC |
| 700 bp | TCGACTGCTAGTGCTTACGGCCGTAGCCGGTCGATCG |
| (SEQ ID | TACGTGCATGCCCTCCCGGTAGTCTCTCGTCGTGCAA |
| NO: 3123) | GCTGCCTCCAGCTTACCAGATTCGATAAAGAGTTTAT |
| | GAGATTTAAGGTCAAGACAACGAGACACGAGTTCGAG |
| | ATTGAGGGAGAGAAGGCCCCTCAGCGGCCTTATAACT |
| | ATAACGGTCCTAAGGTAGCGAACGAACAAACCGCTAA |
| | GCTCAAGGTCACAAAAGGTCGACGAGGACCCGGATCC |
| | CTCCCCTTCTCCTGGTACGGAAGCAAAGCCTATGTTA |
| | AACACTGACTATCTGAAGCTCTCCTTCCCTGAAGGCT |
| | TGAGAGATTCATGAACTTCGAGGAAGGACGGAGAGTT |
| | TATTTATAATGCCATGCGCAATGCTCGCAAATTGGCC |
| | GGTACCGTACTTAACCCGAGTTCAAGCTGAGCCGTTT |
| | CGTTAGCGTGCCGCGCAGCAGCTCGCTCAACGACCCT |
| | CGCTCGTGCGCCTGAGTGCTCCATCTTAGCGTGTACT |
| | GGCTAATAAAACTGGTGCGCCGTAAGGTCCGTGCGAC |
| | TGACTGCCTGTCAAGCACAACTGCTAGCTACTGGAAC |
| | CAACTTCCCCTCCGATGGCCCTGTCATGAATTCT |

### DNA Target Capture

To optimize capture efficiency, two different DNA polymerases (Omni Klentaq LA and Kapa HiFi) were tested in gap filling Mix of the capture step (see table below) with LASSO probes that target 1 Kb and 3 Kb within M13mp18 bacteriphage genome. 3 tenfold increases concentrations in Ampligase DNA Ligase were tested in the components used in the gap filling Mix of the capture step with LASSO probes that target 1 Kb on single stranded and double stranded DNA of the M13mp18 bacteriophage.

Composition of Gap Filling Mix with 0.5 U DNA Ligase in the Reaction Volume (20 μl) and Omni Klentaq LA

| COMPONENT | Amount PER 100 μl Stock |
|---|---|
| Omni Kelntaq gap filling mix | 0.5 U ligase |
| PCR grade Water | 75.1 μl |
| 10X Ampligase DNA ligase Buffer | 10 μl |
| 10 mM dNTPs | 0.4 μl |
| Ampligase DNA Ligase (100 U/μl) | 0.1 μl |
| Omni Klentaq LA | 0.4 μl |
| NADH | 4 μl |
| Glycerol | 10 μl |

Composition of Gap Filling Mixes with DNA Ligase at Various Amount in the Reaction Volume (20 μl) and Kapa HiFi Polymerase

| COMPONENT | Amount PER 100 μl Stock | | |
|---|---|---|---|
| Kapa HiFi gap | 0.5 U ligase | 5 U ligase | 50 U ligase |
| filling mix | | | |
| PCR grade Water | 74.7 μl | 73.7 μl | 64.7 μl |
| 10X Ampligase | 10 μl | 10 μl | 10 μl |
| DNA ligase Buffer | | | |
| 10 mM dNTPs | 0.4 μl | 0.4 μl | 0.4 μl |
| Ampligase DNA | 0.1 μl | l μl | 10 μl |
| Ligase (100 U/μl) | | | |
| Kapa HiFi | 0.8 μl | 0.8 μl | 0.8 μl |
| NADH | 4 μl | 4 μl | 4 μl |
| Glycerol | 10 μl | 10 μl | 10 μl |

The Kapa HiFi based gap filling mix with 5 U ligase in the final reaction volume was used for most of the captures, namely: LASSOs targeting 3 Kb sequences within the single strand M13mp18 DNA 3 different Tm of the ligation arms 65° C., 70° C. and 75° C., with 350 bp and 700 bp backbone linker, LASSOs targeting 4 and 5 Kb sequences within the single strand M13mp18 DNA, and LASSO probes pools that target E. coli DNA and have different melting temperature arms.

The capture was completed with a digestion step after which we performed a post-capture PCR according to the protocol listed in Chkaiban et al. (Curr Protoc, (11):e278, 2021). The primers used in the post capture PCR reaction were AttB1 CaptF (SEQ ID NO: 3110) and AttB2 CaptR (SEQ ID NO: 3111). The total amount of post-capture product was used as an estimate of the efficiency of the capture reaction.

### Sanger Sequencing:

The band from the electrophoresis gel showing a 5 Kb captured target band size from the ssDNA of M13mp18 was excited and purified using Monarch DNA Gel Extraction Kit (#T1020S). Sanger sequencing was performed on the eluate to confirm the identity of the band.

### DNA Preparation and Barcoding of Pools for Oxford Nanopore Sequencing

The ligation kit SQK-LSK 109 was used with the PCR barcoding expansion 1-12 EXP-PBC0001 supplied by Oxford Nanopore and followed the respective protocols for DNA sample preparation for sequencing. An R 9.4.1 flow cell was primed with the component supplied in in the flow cell priming kit (EXP-FLP002) and loaded 50 fmol after mixing it with loading beads and sequencing buffer supplied with the kits. The sequencing was run in the MinION Mk1C and set it for real-time data acquisition and basecalling.

### Sequencing Data Analysis

The resulting reads found in fastq files were aligned and subdivided according to their barcode directly in the MinKNOW app built-in the MinION Mk1C. Each pool was mapped against the ORFeome reference file for Escherichia coli str. k-12 substr. mg1655 found in NCBI (RefSeq: NC_000913.3) uploaded locally as a fasta file. The filtering and the statistical analyses and resulting bean plot graph were performed on R software.

### Cloning the Captured Amplicons Pools in the Gateway System

The post capture PCR product pools were bead purified and mixed with the Gateway ‘donor vectors’ (pDONR221) and the BP Clonase enzyme mix (Invitrogen). The BP reaction was purified and used for electroporation in NEB® 10-beta Electro-competent E. coli (c3020K) to generate cloned libraries. Plasmids were extracted and digested them with EcoRV restriction enzymes to linearize them and proceeded with end repair and DNA preparation for sequencing with the same ligation and barcoding kit used for the amplicon pools mentioned above (SQK-LSK109 with EXP-PBC001).

### Example 8

### Effect of DNA Polymerase Type and Ligase Concentration on Capture Efficiency

DNA polymerase extends the 3′ end starting from the extension arm and copies the target sequence until the ligation arm, where it dissociates allowing the ligation of the 5′-end with the phosphate of the ligation arm. In some examples, a polymerase with low strand displacement is used so it can dissociate when it reaches the ligation arm and give the opportunity for the ligase to close the LASSO. Exemplary polymerases with low strand displacement include the stoffel fragment of the AmpliTaq DNA polymerase (Applied Biosystems), Omni Klentaq LA (DNA polymerase technologies), and Kapa HiFi.

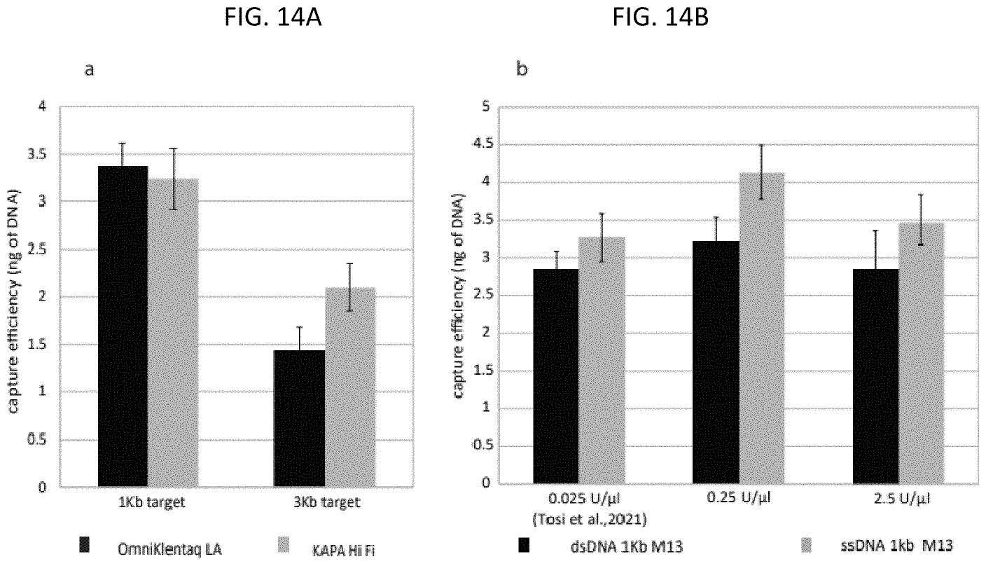

Two different polymerases (Omni Klentaq LA and Kapa HiFi) were analyzed when capturing 1 Kb and 3 Kb target within ds DNA of the M13mp18 phage genome, while all the other components of the gap filling mixes remained the same. Although the two polymerases did not have a significantly different effect on the 1 Kb target capture—estimated in ng of PCR post capture product—Kapa HiFi consistently generated more postcapture PCR products for the longer 3 Kb target capture (FIG. 14A). Thus, Kapa HiFi was used for all subsequent experiments.

The concentration of DNA ligase in the gap filling mix (by 10 fold increases) was determined. Capture on single strand DNA templates produced higher capture efficiency then when starting with double stranded DNA (FIG. 14B). In addition, among the three conditions tested, 5 unit of DNA ligase/20 μl reaction volume was an optimal concentration in terms of efficiency (FIG. 14B). Thus, increasing the concentration of DNA ligase by 10-fold improved target capture efficiency (from 0.5 to 5 U in 20 ul of the reaction volume).

### Example 9

### Effect of DNA Backbone Length and Tm Ligation Arms on Capture Efficiency

The effect of backbone length and ligation arm length on capture efficiency was examined by assembling six LASSO probes having three progressively longer ligation arms for each backbone 350 and 700 bp that targeted the same 3 Kb region on ssDNA of M13mp18 phage. LASSOs with the shorter 350 bp backbone performed better than with longer backbone 700 bp, especially for 1 Kb targets (FIG. 15B lane 1 and 6). Thus the 350 bp backbone for LASSO probe assembly was used for subsequent experiments. FIGS. 15A and 15B show the effect of Tm of the ligation arm on capture efficiency. The highest capture efficiency was obtained when using a ligation arm of 70° C.

### Example 10

### 4 and 5 Kb Target Capture

To test the capability of the LASSO technology in capturing long DNA targets, pre-LASSO probes were designed that target 4 and 5 Kb sequences on single strand M13mp18 genomic DNA with Tm of the extension arms ˜65° C. and the Tm of the ligation arms ˜70° C. When running the post capture PCR product on an electrophoresis gel bands were detected at around 4 kb and 5 kb, indicating successful capture of the targeted sequences (FIG. 15C). Furthermore, the identity of the 5 kb band was corroborated by Sanger sequencing it after excising and purifying it from the gel. The two chromatograms, obtained by sequencing with forward and reverse post capture PCR primers, showed close to the beginnings the presence of a sequence that mapped with ligation (in green) and extension (in red) arms as per the design of the probe (FIG. 15D) followed by the rest of the targeted sequence indicating that the target was captured in its full length.

### Example 11

### Capture Efficiency of LASSO Pools of Different Melting Temperatures Arms

One challenge of the LASSO capture is designing a pool of probes that can capture their targets with similar efficiencies so that in the final captured library all the targets are represented with the similar frequency.

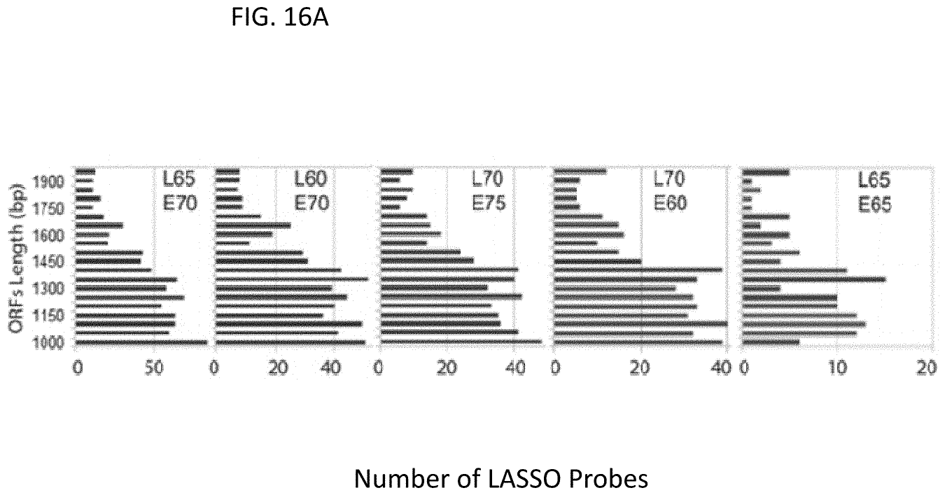

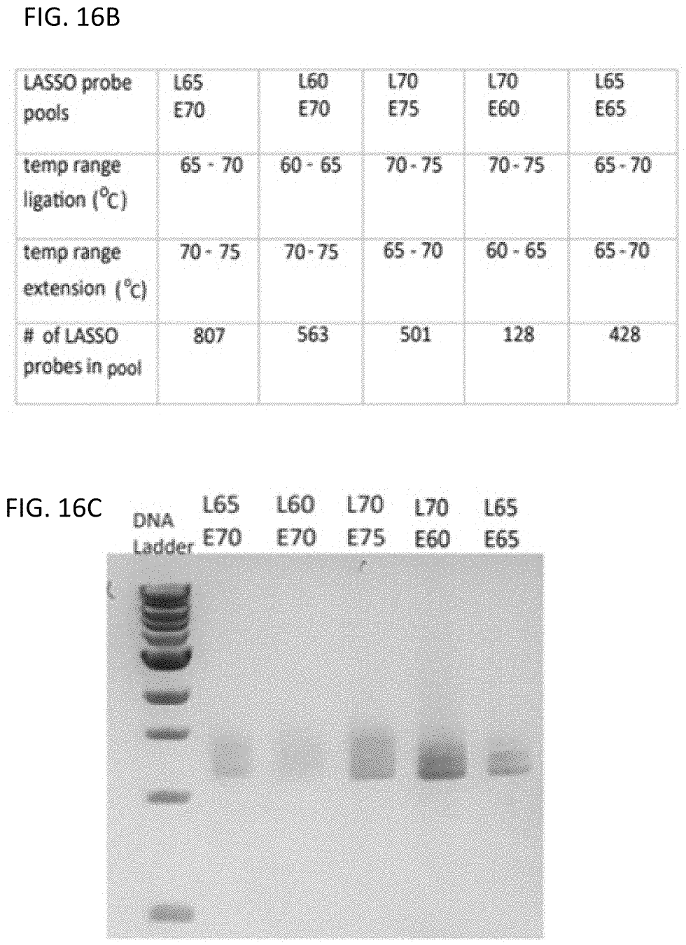

To establish more accurate and improved parameters for the design of pre LASSOs, LASSO pools of varied melting temperature (T_{M}) arms were tested when capturing targets within the E. coli ORFeome from 999 bp-2000 bp. FIG. 16A shows the distribution of the potential targets of the designed LASSOs by length into bins of 50 bp incrementally. Most of the LASSOs target sequences ranged from 1000 to 1400 bp. FIG. 16B lists the T_{M} arm ranges and the number of LASSO the algorithm generated for each pool (as described in Example 7). The algorithm produced 128 to 807 targets/LASSO out of the 4140 ORF for each pool. Running the post capture product on a gel of the various captured pools showed a smear for each pool in the expected size range (FIG. 16C). The smear was more pronounced in the range of 1000 to 1400 bp, in accordance to the size distribution initially produced by the algorithm. The amplicons were sequenced with MinION Mk1C or shuttled into pDNOR vector via Gateway system. The gateway reaction was used for cloning in E. coli and antibiotic resistant colonies were selected from agar plates and the extracted plasmid were sequenced.

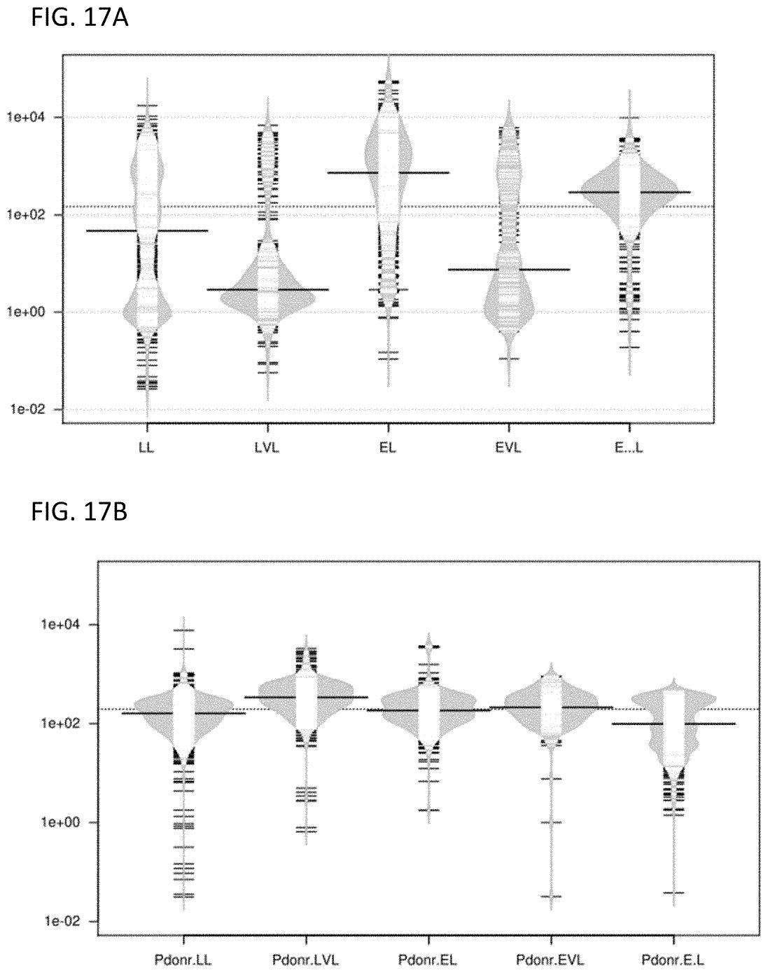

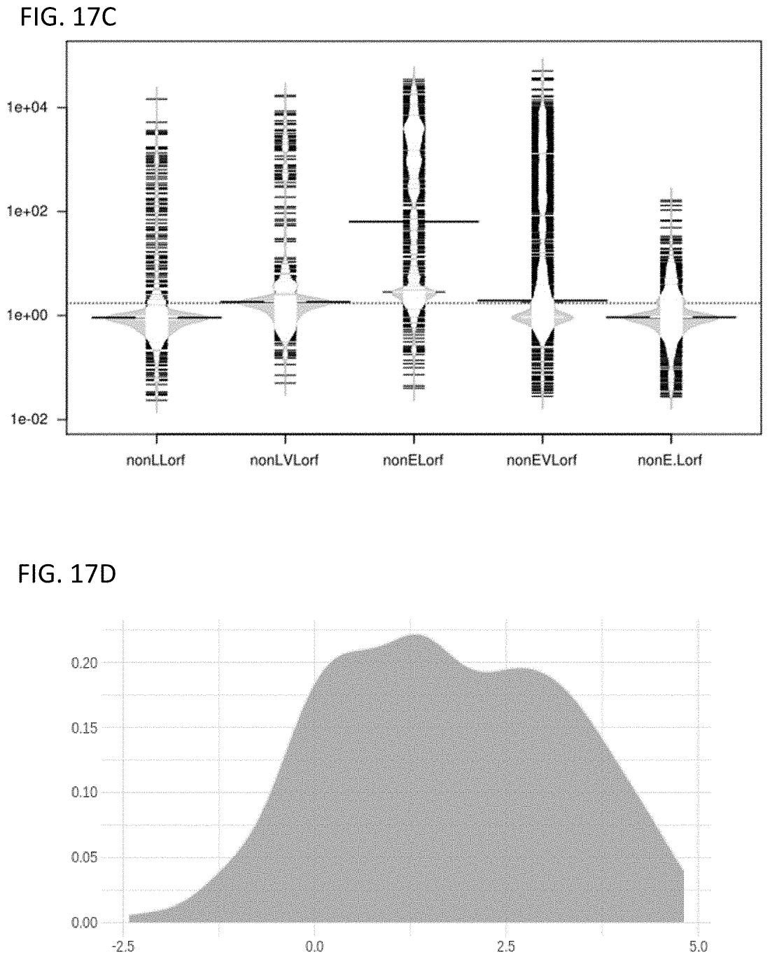

Using R software, the depth of coverage for each target was calculated and plotted it for both the pools of captured amplicons (FIG. 17A) and the pools of the amplicons transformed into pDNOR 221 plasmids (FIG. 17B). With respect to the pools of amplicon targets, the highest coverage on average was obtained for the targeted ORFs captured with the L70E65 pool that had the melting temperature of the extension arm in the range of 65-70° C. and ligation arm in 70-75° C., whereas the most homogeneous distribution was observed in the L65E65 because it yielded the lowest mean log deviation (MLD) of 0.77 in comparison to 2.90, 3.73, 2.06, 3.24 of the L65E70, L60E70, L70E65, L70E60 pools respectively. The mean log deviation (MLD) was used as an indicator dis-proportionality in the coverage of targeted of ORF. When the coverage of non-targeted ORFs was filtered and computed for each pool, the median coverage was 0.91, 1.83, 63.99, 1.94 and 0.93 for L65E70, L60E70, L70E65, L70E60 and L65E65 respectively (FIG. 17C). This shows the low specificity for probes (L70E65 pool) having a ligation arm Tm ˜5° C. higher than the extension arm (65-70° C.), while the highest specificity was for the pool (L65E65) that had extension and ligation arm melting temperature in the same range (65-70° C.), recorded as lowest coverage for untargeted sequences. Thus, the best capture uniformity, in terms of probes representation, highest target enrichment and specificity and almost complete capture of all targets was obtained with probes designed with equal melting arm temperature in the 65-70° C. range.

In addition, at a cutoff of three times the median non-target coverage, around 49.81%, 18.47%, 60.68%, 46.09%, 96.26% of the targeted ORFs were successfully captured for L65E70, L60E70, L70E65, L70E60 and L65E65, respectively, indicating the higher capture efficiency of LASSOs that had similar melting temperature arms at (65-70° C.). In addition, a 57.41, 0.92, 7.60, 4.26 and 315.69-fold enrichment of coverage for captured target versus coverage for captured non targeted ORF's was observed for each of L65E70, L60E70, L70E65, L70E60 and L65E65 pools, respectively.

To further investigate the effect of the difference between melting arm temperature within the pool that had similar extension and ligation arm (65-70° C.) we plotted the ΔTm (Tm extension arm−Tm ligation arm) against data point density and observed a higher density of capture targets when was extension Tm was slightly higher 2.5° C. to equal to the ligation Tm (FIG. 17D). With respect to libraries of the transformed amplicons into pDNOR 221 plasmids, the median coverage for targeted ORF was similar to all the pools (˜199) (FIG. 17B).

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

We claim:

1. A single stranded (ss) DNA Long Adapter Single Stranded Oligonucleotide (LASSO) probe, comprising, from 5′ to 3′:
a ligation arm sequence complementary to a 5′ region of a target sequence;
a backbone sequence that is not complementary to the target sequence, and comprises a recombination site; and
an extension arm sequence complementary to a 3′ region of the target sequence,
wherein the ligation arm sequence and extension arm sequence are complementary to 5′ and 3′ regions of a single target sequence, respectively, and the complementary regions are at least 200 nucleotides (nts) apart on the target sequence.

2. The ssDNA LASSO probe of claim 1, wherein the target sequence is a coding or noncoding DNA sequence.

3. The ssDNA LASSO probe of claim 1, wherein
the ligation arm sequence is about 20 to 50 nts;
the backbone sequence is about 200 to 800 nts;
the extension arm sequence is about 20 to 40 nts; or
combinations thereof.

4. The ssDNA LASSO probe of claim 1, wherein the ssDNA LASSO is about 400 to 800 nts.

5. The ssDNA LASSO probe of claim 1, wherein the target sequence is a single contiguous target sequence.

6. A composition comprising a plurality of the ssDNA LASSO probes of claim 1, wherein the plurality includes oligonucleotides with sequences complementary to at least two different target sequences.

7. A composition comprising:
one or more ssDNA LASSO probes of claim 1, and
a pharmaceutically acceptable carrier.

8. A kit comprising:
one or more ssDNA LASSO probes of claim 1, and
one or more endonucleases, one or more exonucleases, one or more polymerases, one or more ligases, one or more recombinases; one or more reagents for PCR, or combinations thereof.

9. A method of generating the ssDNA LASSO probe of claim 1, comprising:
providing a double stranded pre-LASSO probe comprising from 5′ to 3′(i) a first primer annealing site sequence, (ii) the extension arm sequence, (iii) an inverted PCR primer annealing site comprising a restriction site that allows for asymmetric cutting, (iv) the ligation arm sequence, and (v) a second primer annealing site sequence;
contacting the pre-LASSO probe with a double stranded linear pLASSO vector comprising from 5′ to 3′ (i) the second primer annealing site sequence, (ii) a first backbone region that does not substantially hybridize to the target sequence, (iii) a first recombination site, (iv) a selectable marker, (v) an origin of replication, (vi) a second recombination site, (vii) a second backbone region that does not substantially hybridize to the target sequence, and (viii) the first primer annealing site sequence, wherein the double stranded linear pLASSO vector further includes a nicking endonuclease recognition site, a restriction site not in the backbone, and optionally a first and second restriction endonuclease site, in the presence of a 5′ exonuclease, a polymerase, and a DNA ligase to allow annealing, gap filling and ligation of the first and second primer annealing sites of the pre-LASSO probe to the first and second primer annealing sites of the linear pLASSO vector, thereby generating a circular pLASSO vector containing the pre-LASSO probe;
introducing the circular pLASSO vector into host cells, thereby generating transformed host cells comprising the circular pLASSO vector;
growing the transformed host cells in the presence of a growth media comprising reagents that do not permit growth of the host cells in the absence of the selectable marker;
extracting the circular pLASSO vector from the transformed host cells;
contacting the extracted circular pLASSO vector with a nicking endonuclease specific for the nicking endonuclease recognition site, under conditions that cleave one nucleic acid strand of the extracted circular pLASSO vector, thereby producing a relaxed circular pLASSO vector;
contacting the relaxed circular pLASSO vector with a recombinase specific for the first and second recombination site, under conditions that recombination of the relaxed circular pLASSO vector occurs, thereby generating (i) a plasmid comprising a recombination site, the selection marker, and the origin of replication and (ii) a minicircle comprising the double stranded pre-LASSO probe, the first and second backboned, and a recombination site;
digesting the plasmid with a restriction enzyme and exonuclease V;
using inverted PCR of the minicircle with a first primer and a second primer that hybridize to the inverted PCR primer annealing site, wherein the first primer includes a Type IIS restriction enzyme site and wherein the second primer comprises a 3′-uracil and the first three 5′-end nt are modified nucleotides resistant to exonuclease treatment, thereby generating a linear double stranded minicircle with a 5′ end and 3′ end, wherein the 5′ end of the linear double stranded minicircle is the first primer annealing site at the 3′ end of the linear double stranded minicircle is the second primer annealing site; and
removing all or part of the first and second primer annealing sites from the 5′ and 3′ end of the linear double stranded minicircle by restriction digestion and/or glycosylase digestion; to produce a digested linear double stranded minicircle; and
removing one of the two strands of the digested linear double stranded minicircle, thereby producing the ssDNA LASSO probe.

10. The method of claim 9, wherein removing all or part of the first and second primer annealing sites from the 5′ and 3′ end of the linear double stranded minicircle comprises:
digesting the linear double stranded minicircle with a restriction enzyme that recognizes an asymmetric DNA sequence and cleaves outside its recognition site located in the “inverted PCR primer annealing site” and cleaves the 3′-5′ (bottom strand) a DNA strand exactly at the 5′ end of the extension arm, to produce a digested linear double stranded minicircle′
contacting the digested linear double stranded minicircle with an exonuclease to digest a strand of the digested linear double stranded minicircle that is not protected by the 5′ phosphorothioate bonds, thereby generating a single stranded digested linear double stranded minicircle; and
contacting the single stranded digested linear double stranded minicircle with a USER enzyme, thereby removing all of the first and second primer annealing sites from the 5′ and 3′ end of the linear double stranded minicircle, to generate a mature single strand DNA Lasso probe.

11. The method of claim 9, wherein removing one of the two strands of the digested linear double stranded minicircle comprises incubation with lambda exonuclease.

12. The method of claim 9, wherein providing a double stranded pre-LASSO probe comprises providing a plurality of double stranded pre-LASSO probes, and the method creates a library of ssDNA LASSOs that can target a plurality of sequences.

13. A method of detecting a target sequence, comprising:
contacting a sample comprising the target sequence with the ssDNA LASSO of claim 1, wherein the ligation arm sequence and the extension arm sequence are complimentary to a 5′ region of the target sequence and to a 3′ region of the target sequence, respectively;
hybridizing the ligation arm sequence and extension arm sequence to the target sequence;
gap filling to copy the target sequence between the ligation arm sequence and extension arm sequence using a polymerase;
ligating the resulting molecule, thereby generating a circular single stranded DNA fragment comprising the target sequence;
isolating the circular single-stranded DNA fragment comprising the target sequence; and
amplifying the circular single stranded DNA fragment comprising the target sequences, thereby detecting the target sequences.

14. The method of claim 13, wherein the method detects a plurality of different target sequences, and the method comprises contacting the sample comprising the target sequences with a plurality of ssDNA LASSOs, wherein the plurality of ssDNA LASSOs comprise sequences complementary to the different target sequences.

15. The method of claim 13, wherein the hybridizing and the gap filling are performed at 55-75° C.

16. The method of claim 14, wherein the plurality of different target sequences comprise at least 10,000 different target sequences.

17. The method of claim 14, wherein the sample comprises eukaryotic or prokaryotic genomic DNA (gDNA).

18. The method of claim 17, wherein the gDNA is human gDNA.

19. The method of claim 14, wherein the sample comprises cDNA.

20. A library of target sequences generated by the method of claim 9.

21. A kit, comprising:
a double stranded pre-LASSO probe, comprising from 5′ to 3′(i) a first primer annealing site sequence, (ii) the extension arm sequence, (iii) an inverted PCR primer annealing site comprising a restriction site that allows for asymmetric cutting, (iv) the ligation arm sequence, and (v) a second primer annealing site sequence;
a double stranded linear pLASSO vector comprising from 5′ to 3′ (i) the second primer annealing site sequence (ii) a first backbone region that does not substantially hybridize to the target sequence, (iii) a first recombination site, (iv) a selectable marker, (v) an origin of replication, (vi) a second recombination site, (vii) a second backbone region that does not substantially hybridize to the target sequence, and (viii) the first primer annealing site sequence, wherein the double stranded linear pLASSO vector further includes a nicking endonuclease recognition site, a restriction site not in the backbone, an optional a first restriction endonuclease site and an optional second restriction endonuclease site; and
optionally one or more endonucleases, one or more exonucleases, one or more recombinases; one or more growth media; one or more reagents for inverted PCR, or combinations thereof.
